(19) United States
(12) Patent Application Publication
(10) Pub. No.: US 2014/0161790 A1
(43) Pub. Date: Jun. 12, 2014

(54) ENGINEERED IMMUNOGLOBULINS WITH EXTENDED IN VIVO HALF-LIFE

(71) Applicant: Xencor, Inc, Monrovia, CA (US)

(72) Inventors: John Desjarlais, Pasadena, CA (US); Gregory Alan Lazar, Arcadia, CA (US)

(73) Assignee: Xencor, Inc., Monrovia, CA (US)

(21) Appl. No.: 14/084,515

(22) Filed: Nov. 19, 2013

Related U.S. Application Data
(60) Provisional application No. 61/727,906, filed on Nov. 19, 2012.

(51) Int. Cl. C07K 16/44

(52) U.S. Cl. CPC C07K 16/44
USPC 424/130.1; 530/387.1; 536/23.53; 435/326; 435/320.1

(57) ABSTRACT

The present application relates to immunoglobulin compositions with improved half-life, and their application, particularly for therapeutic purposes.

This application claims the benefit under 35 U.S.C. 119 to U.S. Provisional Application No. 61/727,906, filed Nov. 19, 2012, which is incorporated herein by reference in its entirety.

### FIELD OF THE INVENTION

The present application relates to immunoglobulin compositions with improved half-life, and their application, particularly for therapeutic purposes.

### BACKGROUND OF THE INVENTION

Antibodies are immunological proteins that each binds a specific antigen. In most mammals, including humans and mice, antibodies are constructed from paired heavy and light polypeptide chains. Each chain is made up of individual immunoglobulin (Ig) domains, and thus the generic term immunoglobulin is used for such proteins. Each chain is made up of two distinct regions, referred to as the variable and constant regions. The light and heavy chain variable regions show significant sequence diversity between antibodies, and are responsible for binding the target antigen. The constant regions show less sequence diversity, and are responsible for binding a number of natural proteins to elicit important biochemical events. In humans there are five different classes of antibodies including IgA (which includes subclasses IgA1 and IgA2), IgD, IgE, IgG (which includes subclasses IgG1, IgG2, IgG3, and IgG4), and IgM. The distinguishing feature between these antibody classes is their constant regions, although subtler differences may exist in the V region. IgG antibodies are tetrameric proteins composed of two heavy chains and two light chains. The IgG heavy chain is composed of four immunoglobulin domains linked from N- to C-terminus in the order VH—CH1-CH2-CH3, referring to the heavy chain variable domain, heavy chain constant domain 1, heavy chain constant domain 2, and heavy chain constant domain 3 respectively (also referred to as VH—Cγ1-Cγ2-Cγ3, referring to the heavy chain variable domain, constant gamma 1 domain, constant gamma 2 domain, and constant gamma 3 domain respectively). The IgG light chain is composed of two immunoglobulin domains linked from N- to C-terminus in the order VL-CL, referring to the light chain variable domain and the light chain constant domain respectively.

The neonatal Fc receptor (FcRn) protects IgG from degradation and is therefore responsible in part for the long half-life (˜21 days for IgG1) of antibodies in circulation. FcRn is a heterodimer of a 50 kD α-chain and an 18 kD β2-microglobulin chain, and binds to IgG in the interface between the CH2 and CH3 domains (Burmeister W P et al., 1994, Nature 372:336-343; Martin W L et al., 2001, Molecular cell 7:867-877). IgG protection from degradation occurs via a pH-dependent mechanism of pinocytosis and endosomal recycling. FcRn binds IgG at the lower pH of the early endosome (6-6.5) but not at the higher pH of blood (7.4), a property mediated to a large extent by histidines at the antibody/receptor interface. Endosomal IgG/FcRn binding salvages IgG from lysosomal degradation, as evidenced by the short half-life of IgG in FcRn-deficient mice (Ghetie V et al., 1996, Eur J Immunol 26:690-696) and the rapid turnover of antibodies with mutations that disrupt receptor binding (Vaccaro C et al., 2005, Nature Biotechnology 23:1283-1288; Ward E S et al., 2003, International immunology 15:187-195.

Antibodies have been developed for therapeutic use. Representative publications related to such therapies include Chamow et al., 1996, Trends Biotechnol 14:52-60; Ashkenazi et al., 1997, Curr Opin Immunol 9:195-200, Cragg et al., 1999, Curr Opin Immunol 11:541-547; Glennie et al., 2000, Immunol Today 21:403-410, McLaughlin et al., 1998, J Clin Oncol 16:2825-2833, and Cobleigh et al., 1999, J Clin Oncol 17:2639-2648, all entirely incorporated by reference.

The administration of antibodies and Fc fusion proteins as therapeutics requires injections with a prescribed frequency relating to the clearance and half-life characteristics of the protein. Longer in vivo half-lives allow more seldom injections or lower dosing, which is clearly advantageous. Although the past mutations in the Fc domain have lead to some proteins with increased FcRn binding affinity and in vivo half-lives, these mutations have not identified the optimal mutations and enhanced in vivo half-life. Moreover, although prior work with engineered Fc variants has shown that antibodies with increased binding to the neonatal Fc receptor FcRn at the lower pH of endosomes can have longer half-life in vivo, no studies have demonstrated that such antibodies retained efficacy at longer dosing intervals. For half-life extension technologies to be of practical use, efficacy of a biotherapeutic with longer half-life must be preserved at longer dosing intervals. Although the relationship between drug exposure and efficacy is well-established for small molecules, this correlation has not thus far been established for antibodies that were FcRn-engineered for longer half-life. The present application meets these and other needs.

### SUMMARY OF THE INVENTION

The present application is directed to immunoglobulin compositions with long in vivo half-life. The immunoglobulin compositions of the invention comprise Fc variants of a parent Fc polypeptide, including at least one modification in the Fc region of the polypeptide.

In various embodiments, the variant polypeptides exhibit altered binding to FcRn as compared to a parent polypeptide. In certain variations, the modification can be selected from the group consisting of: 252Y, 254T, 256E, 259I, 308F, 428L, and 434S, where the numbering is according to the EU Index in Kabat et al.

In another embodiment, the Fc variant is selected from the group consisting of: 259I/308F, 252Y/254T/256E, 428L/434S, and 259I/308F/428L.

In preferred embodiments, the immunoglobulins of the invention comprise Fc regions that are variants of human IgG1, IgG2, IgG3, or IgG4 sequences. In certain embodiments, the immunoglobulins of the invention comprise variant Fc regions that are encoded by the amino acid sequences in SEQ ID's 13-19.

The immunoglobulins of the invention are antibodies or immunoadhesins. In preferred embodiments, the antibodies or immunoadhesins of the invention have specificity for an antigen selected from the group consisting of VEGF, TNF, Her2, EGFR, NGF, CD20, IgE, RSV, IL-6R, B7.1 (CD80), and B7.2 (CD86).

In preferred embodiments, the antibodies of the invention comprise variable regions or CDRs encoded by the amino acid sequences in SEQ ID's 20-130. In alternately preferred embodiments, the immunoadhesins comprise fusion partners encoded by the amino acid sequences in SEQ ID's 131-133.

In another embodiment, the invention includes a method of treating a patient in need of said treatment comprising administering an effective amount of an immunogloublin described herein.

### BRIEF DESCRIPTION OF THE DRAWINGS

FIG. 1. Engineered anti-VEGF (bevacizumab) variants increase binding to human FcRn. (a) The log of the equilibrium association constant K_{A} (1/K_{D} obtained from Table 1) at pH 6.0 are plotted for each variant. This binding study used a format in which FcRn analyte bound antigen-captured antibody. IgG1 represents the parent bevacizumab native IgG1 antibody. (b) Illustration of binding sensorgrams at pH 6.0 and 7.4. Antibody as the analyte was bound to an FcRn-coupled chip at pH 6.0 in the association phase, followed by buffer wash at pH 6.0 in the dissociation phase, and then buffer wash at pH 7.4.

FIG. 2. Increasing antibody affinity to FcRn promotes half-life extension in hFcRn mice. (a) Log-linear serum concentration versus time profiles of anti-VEGF antibodies in hFcRn mice. All antibodies were administered via single i.v. bolus at 2 mg/kg, and serum antibody concentrations were determined using a human immunoglobulin recognition immunoassay. Results are plotted as mean±standard error (N=6). IgG1 represents the parent bevacizumab native IgG1. (b) Log-linear serum concentration versus time profiles of anti-EGFR antibodies in hFcRn mice. The study design was identical to that described in panel (a) except that serum concentrations were measured with an EGFR antigen-down immunoassay. IgG1 represents cetuximab C225, and LS represents the Fc engineered version of humanized cetuximab huC225. (c) Correlation plot describing the log-linear relationship between FcRn association and half-life in hFcRn mice (Studies M1-M3). PK parameters obtained from these studies are reported in Table 2, and FcRn affinities (K_{A}'s) for both anti-VEGF and anti-EGFR antibodies are as measured for bevacizumab antibodies (Table 1). Symbols are as in panels (a) and (b).

FIG. 3. Increasing antibody affinity to FcRn promotes half-life extension in cynomolgus monkeys. (a) Log-linear serum concentration versus time profiles of anti-VEGF (bevacizumab) antibodies in cynomolgus monkeys. All antibodies were administered via single 60 minute i.v. infusion at 4 mg/kg and serum antibody concentrations were determined using a VEGF antigen-down immunoassay. Results are shown as mean±standard error (N=2 for bevacizumab and N=3 for variants). (b) Log-linear serum concentration versus time profiles of anti-EGFR antibodies in cynomolgus monkeys. C225 IgG1 and huC225 LS were administered via single 30 minute i.v. infusion at 7.5 mg/kg and serum antibody concentrations were determined using a EGFR antigen-down immunoassay. Results are shown as mean of N=2 animals per test article.

FIG. 4. Improved half-life translates into greater in vivo efficacy. (a) Xenograft study in hFcRn/Rag1^{-/-} mice comparing activity of WT IgG1 and LS variant versions of bevacizumab against established SKOV-3 tumors. Tumor volume is plotted versus day post tumor cell injection. Antibodies were dosed every 10 days starting on day 35 (indicated by the arrows). N=8 mice/group. * p=0.028 at 84 days. (b) Scatter plot of serum antibody concentrations measured for each individual mouse on the final day of data acquisition. (c) Xenograft study in hFcRn/Rag1^{-/-} mice comparing activity of C225 IgG1 and huC225 LS versions of anti-EGFR against established A431 tumors. Tumor volume is plotted versus day post tumor cell injection. Antibodies were dosed every 10 days starting on day 10 (indicated by the arrows). N=9 mice/group. * p=0.005 at 35 days. (d) Scatter plot of serum antibody concentrations measured for each individual mouse on the final day of data acquisition.

FIG. 5. Sequence alignments of human IgG constant heavy chains. Gray indicates differences from IgG1, and boxed residues indicate common allotypic variations in the human population.

FIG. 6. Amino acid sequences of constant regions.

FIG. 7. Amino acid sequences of exemplary Fc regions.

FIG. 8. Amino acid sequences of exemplary variant Fc regions.

FIG. 9. Amino acid sequences of VH and VL variable regions.

FIG. 10. Amino acid sequences of immunoadhesin fusion partners.

FIG. 11. Biacore sensorgrams for binding of anti-TNF antibodies to human FcRn.

FIG. 12. Affinities of anti-TNF antibodies for human FcRn and human TNF as determined by Biacore.

### DETAILED DESCRIPTION OF THE INVENTION

The present invention discloses the generation of novel variants of Fc domains, including those found in antibodies, Fc fusions, and immuno-adhesions, which have an increased binding to the FcRn receptor. As noted herein, binding to FcRn results in longer serum retention in vivo.

In order to increase the retention of the Fc proteins in vivo, the increase in binding affinity must be at around pH 6 while maintaining lower affinity at around pH 7.4. Although still under examination, Fc regions are believed to have longer half-lives in vivo, because binding to FcRn at pH 6 in an endosome sequesters the Fc (Ghetie and Ward, 1997 Immunol Today. 18 (12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall' Acqua et al. showed that Fc mutants with increased FcRn binding at pH 6 and pH 7.4 actually had reduced serum concentrations and the same half life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4.

An additional aspect of the invention is the increase in FcRn binding over wild type specifically at lower pH, about pH 6.0, to facilitate Fc/FcRn binding in the endosome. Also disclosed are Fc variants with altered FcRn binding and altered binding to another class of Fc receptors, the FcγR's (sometimes written FcgammaR's) as differential binding to FcγR5, particularly increased binding to FcγRIIIb and decreased binding to FcγRIIb, has been shown to result in increased efficacy.

### DEFINITIONS

In order that the application may be more completely understood, several definitions are set forth below. Such definitions are meant to encompass grammatical equivalents.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence.

By “amino acid substitution” or “substitution” herein is meant the replacement of an amino acid at a particular position in a parent polypeptide sequence with another amino acid. For example, the substitution N434S refers to a variant polypeptide, in this case an Fc variant, in which the asparagine at position 434 is replaced with serine.

By “amino acid insertion” or “insertion” as used herein is meant the addition of an amino acid sequence at a particular position in a parent polypeptide sequence. For example, −233E or ̂233E designates an insertion of glutamic acid after position 233 and before position 234. Additionally, −233ADE or ̂233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233# designates a deletion of glutamic acid at position 233. Additionally, EDA233- or EDA233# designates a deletion of the sequence GluAspAla that begins at position 233.

By “IgG subclass modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification. By “non-naturally occurring modification” as used herein is meant an amino acid modification that is not isotypic. For example, because none of the IgGs comprise a serine at position 434, the substitution 434S in IgG1, IgG2, IgG3, or IgG4 is considered a non-naturally occurring modification.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. The protein variant sequence herein will preferably possess at least about 80% homology with a parent protein sequence, and most preferably at least about 90% homology, more preferably at least about 95% homology. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising a modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S. A relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention, numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and non-naturally occurring amino acids. Variants may comprise non-natural amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

By “amino acid” and “amino acid identity” as used herein is meant one of the 20 naturally occurring amino acids or any non-natural analogues that may be present at a specific, defined position.

By “effector function” as used herein is meant a biochemical event that results from the interaction of an antibody Fc region with an Fc receptor or ligand. Effector functions include but are not limited to “antibody dependent cell-mediated cytotoxicity (ADCC), antibody dependent cell-mediated phagocytosis (ADCP), and complement dependent cytotoxicity (CDC).

By “IgG Fc ligand” as used herein is meant a molecule, preferably a polypeptide, from any organism that binds to the Fc region of an IgG antibody to form an Fc/Fc ligand complex. Fc ligands include but are not limited to FcγR5, FcγR5, FcγR5, FcRn, G1g, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγR5 (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγR5 or FcγR isoforms or allotypes. An FcγR may be from any organism, including but not limited to humans, mice, rats, rabbits, and monkeys. Mouse FcγR5 include but are not limited to FcγRI (CD64), FcγRII (CD32), FcγRIII (CD16), and FcγRIII-2 (CD16-2), as well as any undiscovered mouse FcγR5 or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless other wise noted herein, FcRn or an FcRn protein refers to the complex of FcRn heavy chain with beta-2-microglobulin. Sequences of particular interest of FcRn are shown in the Figures, particularly the human species.

By “clearance” as used herein is meant the volume of body fluid from which the antibody or immunoadhesin is, apparently, completely removed by biotransformation and/or excretion, per unit time. In fact, the antibody or immunoadhesin is only partially removed from each unit volume of the total volume in which it is dissolved. Since the concentration of the antibody or immunoadhesin in its volume of distribution is most commonly sampled by analysis of blood or plasma, clearances are most commonly described as the “plasma clearance” or “blood clearance” of a substance.

By “half-life” as used herein is meant the period of time for a substance undergoing decay, to decrease by half. For an antibody or immunoadhesin, half-life refers to its pharmacokinetic properties in vivo. In this context, the half-life is the period of time for the serum concentration of an antibody or immunoadhesion to decrease by half.

By “parent polypeptide” as used herein is meant an unmodified polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “position” as used herein is meant a location in the sequence of a protein. Positions may be numbered sequentially, or according to an established format, for example the EU index for antibody numbering.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89 (20):9367 (1992), entirely incorporated by reference). The amino acids may either be naturally occurring or non-naturally occurring; as will be appreciated by those in the art. For example, homo-phenylalanine, citrulline, and noreleucine are considered amino acids for the purposes of the invention, and both D- and L- (R or S) configured amino acids may be utilized. The variants of the present invention may comprise modifications that include the use of unnatural amino acids incorporated using, for example, the technologies developed by Schultz and colleagues, including but not limited to methods described by Cropp & Shultz, 2004, Trends Genet. 20 (12):625-30, Anderson et al., 2004, Proc Natl Acad Sci USA 101 (2):7566-71, Zhang et al., 2003, 303 (5656):371-3, and Chin et al., 2003, Science 301 (5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “target antigen” as used herein is meant the molecule that is bound specifically by the variable region of a given antibody. A target antigen may be a protein, carbohydrate, lipid, or other chemical compound.

By “target cell” as used herein is meant a cell that expresses a target antigen.

By “variable region” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The present invention is directed to antibodies that exhibit increased binding to FcRn relative to a wild-type antibody. For example, in some instances, increased binding results in cellular recycling of the antibody and hence increased half-life. In addition, antibodies exhibiting increased binding to FcRn and altered binding to other Fc receptors, eg. FcγRs, find use in the present invention.

Antibodies

The present application is directed to antibodies that include amino acid modifications that modulate binding to FcRn. Of particular interest are antibodies that minimally comprise an Fc region, or functional variant thereof, that displays increased binding affinity to FcRn at lowered pH, and do not exhibit substantially altered binding at higher pH.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE.

The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

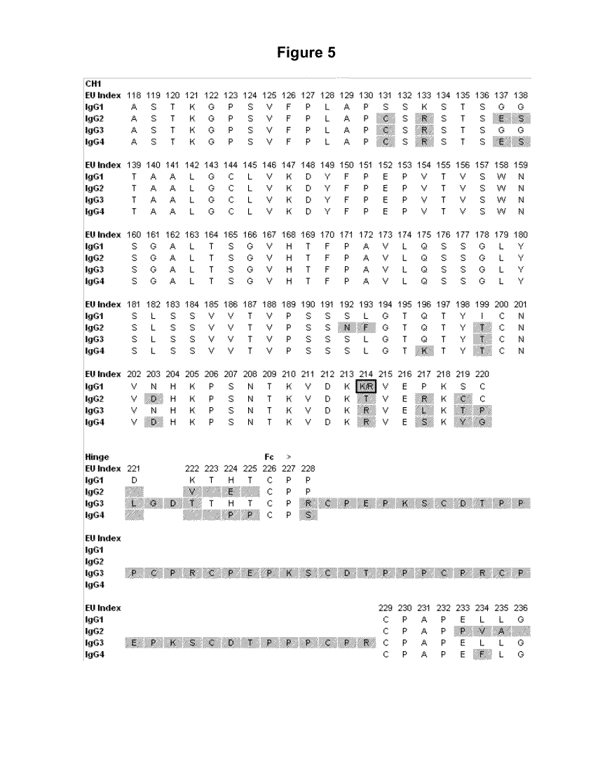

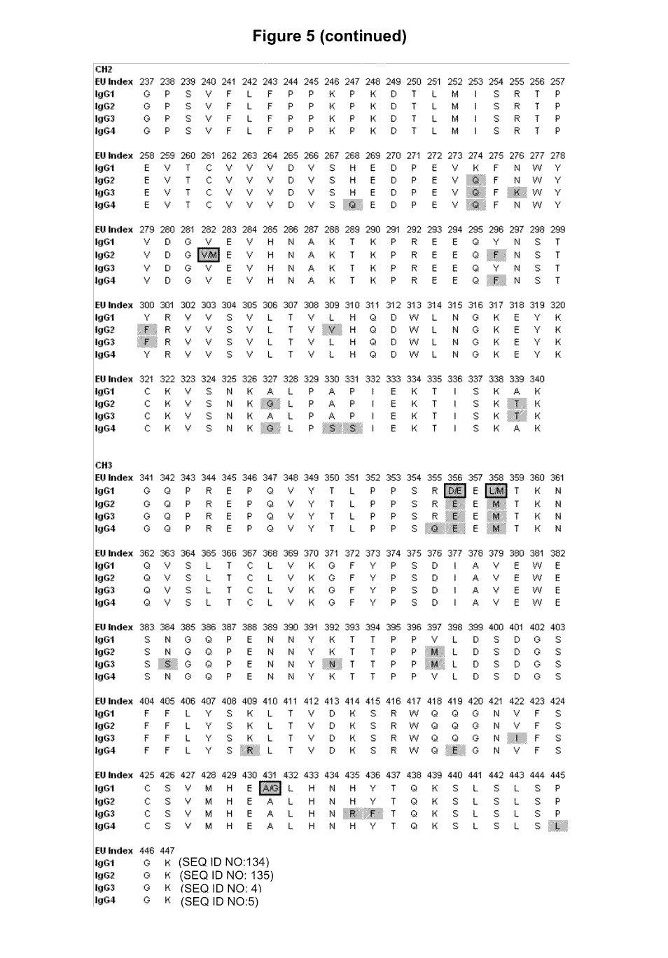

Of particular interest in the present invention are the Fc regions. By “Fc” or “Fc region”, as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, as illustrated in FIG. 5, Fc comprises immunoglobulin domains Cgamma2 and Cgamma3 (Cg2 and Cg3) and the lower hinge region between Cgamma1 (Cg1) and Cgamma2 (Cg2). Although the boundaries of the Fc region may vary, the human IgG heavy chain Fc region is usually defined to include residues C226 or P230 to its carboxyl-terminus, wherein the numbering is according to the EU index as in Kabat. Fc may refer to this region in isolation, or this region in the context of an Fc polypeptide, as described below. By “Fc polypeptide” as used herein is meant a polypeptide that comprises all or part of an Fc region. Fc polypeptides include antibodies, Fc fusions, isolated Fcs, and Fc fragments.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively.

Chimeric and Humanized Antibodies

In some embodiments, the scaffold components can be a mixture from different species. As such, if the protein is an antibody, such antibody may be a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57 (20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272 (16):10678-10684; Rosok et al., 1996, J. Biol. Chem. 271 (37): 22611-22618; Rader et al., 1998, Proc. Natl. Acad. Sci. USA 95: 8910-8915; Krauss et al., 2003, Protein Engineering 16 (10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

Antibody Fusions

In one embodiment, the antibodies of the invention are antibody fusion proteins (sometimes referred to herein as an “antibody conjugate”). One type of antibody fusions comprises Fc fusions, which join the Fc region with a conjugate partner. By “Fc fusion” as used herein is meant a protein wherein one or more polypeptides is operably linked to an Fc region. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200, both entirely incorporated by reference). An Fc fusion combines the Fc region of an immunoglobulin with a fusion partner, which in general can be any protein or small molecule. Virtually any protein or small molecule may be linked to Fc to generate an Fc fusion. Protein fusion partners may include, but are not limited to, the variable region of any antibody, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target. Such targets may be any molecule, preferably an extracellular receptor, which is implicated in disease. Thus, the IgG variants can be linked to one or more fusion partners. In one alternate embodiment, the IgG variant is conjugated or operably linked to another therapeutic compound. The therapeutic compound may be a cytotoxic agent, a chemotherapeutic agent, a toxin, a radioisotope, a cytokine, or other therapeutically active agent. The IgG may be linked to one of a variety of nonproteinaceous polymers, e.g., polyethylene glycol, polypropylene glycol, polyoxyalkylenes, or copolymers of polyethylene glycol and polypropylene glycol.

In addition to Fc fusions, antibody fusions include the fusion of the constant region of the heavy chain with one or more fusion partners (again including the variable region of any antibody), while other antibody fusions are substantially or completely full length antibodies with fusion partners. In one embodiment, a role of the fusion partner is to mediate target binding, and thus it is functionally analogous to the variable regions of an antibody (and in fact can be). Virtually any protein or small molecule may be linked to Fc to generate an Fc fusion (or antibody fusion). Protein fusion partners may include, but are not limited to, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target. Such targets may be any molecule, preferably an extracellular receptor, which is implicated in disease.

The conjugate partner can be proteinaceous or non-proteinaceous; the latter generally being generated using functional groups on the antibody and on the conjugate partner. For example linkers are known in the art; for example, homo- or hetero-bifunctional linkers as are well known (see, 1994 Pierce Chemical Company catalog, technical section on cross-linkers, pages 155-200, incorporated herein by reference).

Suitable conjugates include, but are not limited to, labels as described below, drugs and cytotoxic agents including, but not limited to, cytotoxic drugs (e.g., chemotherapeutic agents) or toxins or active fragments of such toxins. Suitable toxins and their corresponding fragments include diptheria A chain, exotoxin A chain, ricin A chain, abrin A chain, curcin, crotin, phenomycin, enomycin and the like. Cytotoxic agents also include radiochemicals made by conjugating radioisotopes to antibodies, or binding of a radionuclide to a chelating agent that has been covalently attached to the antibody. Additional embodiments utilize calicheamicin, auristatins, geldanamycin, maytansine, and duocarmycins and analogs; for the latter, see U.S. 2003/0050331A1, entirely incorporated by reference.

Antibody Fragments

In one embodiment, the antibody is an antibody fragment. Of particular interest are antibodies that comprise Fc regions, Fc fusions, and the constant region of the heavy chain (CH1-hinge-CH2-CH3), again also including constant heavy region fusions.

Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference). The antibody fragments may be modified. For example, the molecules may be stabilized by the incorporation of disulphide bridges linking the VH and VL domains (Reiter et al., 1996, Nature Biotech. 14:1239-1245, entirely incorporated by reference).

IgG Variants

In one embodiment, the invention provides variant IgG proteins. At a minimum, IgG variants comprise an antibody fragment comprising the CH2-CH3 region of the heavy chain. In addition, suitable IgG variants comprise Fc domains (e.g. including the lower hinge region), as well as IgG variants comprising the constant region of the heavy chain (CH1-hinge-CH2-CH3) also being useful in the present invention, all of which can be fused to fusion partners.

An IgG variant includes one or more amino acid modifications relative to a parent IgG polypeptide, in some cases relative to the wild type IgG. The IgG variant can have one or more optimized properties. An IgG variant differs in amino acid sequence from its parent IgG by virtue of at least one amino acid modification. Thus IgG variants have at least one amino acid modification compared to the parent. Alternatively, the IgG variants may have more than one amino acid modification as compared to the parent, for example from about one to fifty amino acid modifications, preferably from about one to ten amino acid modifications, and most preferably from about one to about five amino acid modifications compared to the parent.

Thus the sequences of the IgG variants and those of the parent Fc polypeptide are substantially homologous. For example, the variant IgG variant sequences herein will possess about 80% homology with the parent IgG variant sequence, preferably at least about 90% homology, and most preferably at least about 95% homology. Modifications may be made genetically using molecular biology, or may be made enzymatically or chemically.

The present application also provides IgG variants that are optimized for a variety of therapeutically relevant properties. An IgG variant that is engineered or predicted to display one or more optimized properties is herein referred to as an “optimized IgG variant”. The most preferred properties that may be optimized include but are not limited to enhanced or reduced affinity for FcRn and increased or decreased in vivo half-life. Suitable embodiments include antibodies that exhibit increased binding affinity to FcRn at lowered pH, such as the pH associated with endosomes, e.g. pH 6.0, while maintaining the reduced affinity at higher pH, such as 7.4., to allow increased uptake into endosomes but normal release rates. Preferred variants are described in U.S. Ser. No. 12/341,769. Similarly, these antibodies with modulated FcRn binding may optionally have other desirable properties, such as modulated FcγR binding, such as outlined in U.S. Ser. Nos. 11/174,287, 11/124,640, 10/822,231, 10/672,280, 10/379,392, and the patent application entitled IgG Immunoglobulin variants with optimized effector function filed on Oct. 21, 2005 having application Ser. No. 11/256,060.

Methods of Using IgG Variants

The IgG variants may find use in a wide range of products. In one embodiment the IgG variant is a therapeutic, a diagnostic, or a research reagent, preferably a therapeutic. The IgG variant may find use in an antibody composition that is monoclonal or polyclonal. In a preferred embodiment, the IgG variants are used to kill target cells that bear the target antigen, for example cancer cells. In an alternate embodiment, the IgG variants are used to block, antagonize or agonize the target antigen, for example for antagonizing a cytokine or cytokine receptor. In an alternately preferred embodiment, the IgG variants are used to block, antagonize or agonize the target antigen and kill the target cells that bear the target antigen.

The IgG variants may be used for various therapeutic purposes. In a preferred embodiment, an antibody comprising the IgG variant is administered to a patient to treat an antibody-related disorder. A “patient” for the purposes includes humans and other animals, preferably mammals and most preferably humans. By “antibody related disorder” or “antibody responsive disorder” or “condition” or “disease” herein are meant a disorder that may be ameliorated by the administration of a pharmaceutical composition comprising an IgG variant. Antibody related disorders include but are not limited to autoimmune diseases, immunological diseases, infectious diseases, inflammatory diseases, neurological diseases, and oncological and neoplastic diseases including cancer. By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia and lymphoid malignancies.

In one embodiment, an IgG variant is the only therapeutically active agent administered to a patient. Alternatively, the IgG variant is administered in combination with one or more other therapeutic agents, including but not limited to cytotoxic agents, chemotherapeutic agents, cytokines, growth inhibitory agents, anti-hormonal agents, kinase inhibitors, anti-angiogenic agents, cardioprotectants, or other therapeutic agents. The IgG variants may be administered concomitantly with one or more other therapeutic regimens. For example, an IgG variant may be administered to the patient along with chemotherapy, radiation therapy, or both chemotherapy and radiation therapy. In one embodiment, the IgG variant may be administered in conjunction with one or more antibodies, which may or may not be an IgG variant. In accordance with another embodiment, the IgG variant and one or more other anti-cancer therapies are employed to treat cancer cells ex vivo. It is contemplated that such ex vivo treatment may be useful in bone marrow transplantation and particularly, autologous bone marrow transplantation. It is of course contemplated that the IgG variants can be employed in combination with still other therapeutic techniques such as surgery.

A variety of other therapeutic agents may find use for administration with the IgG variants. In one embodiment, the IgG is administered with an anti-angiogenic agent. By “anti-angiogenic agent” as used herein is meant a compound that blocks, or interferes to some degree, the development of blood vessels. The anti-angiogenic factor may, for instance, be a small molecule or a protein, for example an antibody, Fc fusion, or cytokine, that binds to a growth factor or growth factor receptor involved in promoting angiogenesis. The preferred anti-angiogenic factor herein is an antibody that binds to Vascular Endothelial Growth Factor (VEGF). In an alternate embodiment, the IgG is administered with a therapeutic agent that induces or enhances adaptive immune response, for example an antibody that targets CTLA-4. In an alternate embodiment, the IgG is administered with a tyrosine kinase inhibitor. By “tyrosine kinase inhibitor” as used herein is meant a molecule that inhibits to some extent tyrosine kinase activity of a tyrosine kinase. In an alternate embodiment, the IgG variants are administered with a cytokine.

Pharmaceutical compositions are contemplated wherein an IgG variant and one or more therapeutically active agents are formulated. Formulations of the IgG variants are prepared for storage by mixing the IgG having the desired degree of purity with optional pharmaceutically acceptable carriers, excipients or stabilizers (Remington's Pharmaceutical Sciences 16th edition, Osol, A. Ed., 1980, entirely incorporated by reference), in the form of lyophilized formulations or aqueous solutions. The formulations to be used for in vivo administration are preferably sterile. This is readily accomplished by filtration through sterile filtration membranes or other methods. The IgG variants and other therapeutically active agents disclosed herein may also be formulated as immunoliposomes, and/or entrapped in microcapsules.

The concentration of the therapeutically active IgG variant in the formulation may vary from about 0.1 to 100% by weight. In a preferred embodiment, the concentration of the IgG is in the range of 0.003 to 1.0 molar. In order to treat a patient, a therapeutically effective dose of the IgG variant may be administered. By “therapeutically effective dose” herein is meant a dose that produces the effects for which it is administered. The exact dose will depend on the purpose of the treatment, and will be ascertainable by one skilled in the art using known techniques. Dosages may range from 0.01 to 100 mg/kg of body weight or greater, for example 0.01, 0.1, 1.0, 10, or 50 mg/kg of body weight, with 1 to 10 mg/kg being preferred. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Administration of the pharmaceutical composition comprising an IgG variant, preferably in the form of a sterile aqueous solution, may be done in a variety of ways, including, but not limited to, orally, subcutaneously, intravenously, parenterally, intranasally, intraotically, intraocularly, rectally, vaginally, transdermally, topically (e.g., gels, salves, lotions, creams, etc.), intraperitoneally, intramuscularly, intrapulmonary (e.g., AERx® inhalable technology commercially available from Aradigm, or Inhance® pulmonary delivery system commercially available from Nektar Therapeutics, etc.). Therapeutic described herein may be administered with other therapeutics concomitantly, i.e., the therapeutics described herein may be co-administered with other therapies or therapeutics, including for example, small molecules, other biologicals, radiation therapy, surgery, etc.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence. For all variable region positions discussed in the present invention, numbering is according to the Kabat numbering scheme (Kabat et al., 1991, Sequences of Proteins of Immunological Interest, 5th Ed., United States Public Health Service, National Institutes of Health, Bethesda, entirely incorporated by reference).

### Example 1

### Engineered Variants Improve Affinity for FcRn at pH 6.0

Rational design methods coupled with high-throughput protein screening were used to engineer a series of Fc variants with greater affinity for human FcRn. Variants were constructed in the context of the humanized anti-VEGF IgG1 antibody bevacizumab (Presta L G et al., 1997, Cancer Research 57, 4593-4599) (Avastin®, Genentech/Roche), which is currently approved for the treatment of colorectal, lung, breast, and renal cancers.

Genes encoding antibody heavy and light chains were contructed in the mammalian expression vector pTT5 (NRC-BRI, Canada) (Durocher Y et al., 2002, Nucleic acids research 30:E9). Human gamma and CK constant chain genes were obtained from IMAGE clones, and variable region genes encoding the anti-VEGF VH and VL domains were synthesized commercially (Blue Heron Biotechnologies). Variable region genes encoding cetuximab and humanized cetuximab have been described previously (Naramura M et al., 1993, Cancer Immunol Immunother 37:343-349; Lazar G A et al., 2007, Molecular Immunology 44:1986-1998). Fc mutations were constructed using the QuikChange® site-directed mutagenesis (Agilent). All DNA was sequenced to confirm the fidelity of the sequences. Plasmids containing heavy and light chain genes were co-transfected into HEK293E cells (Durocher Y et al., 2002, Nucleic Acids Research 30:E9) using lipofectamine and grown in FreeStyle 293 media (Invitrogen). After 5 days of growth, the antibodies were purified from the culture supernatant by protein A affinity using MabSelect resin (GE Healthcare) and formulated in calcium- and magnesium-free PBS.

Genes encoding the α- and β2-microglobulin chains of hFcRn were PCR-amplified from cDNA clones and cloned into the vector pcDNA3.1Zeo (both from Invitrogen). The chains were co-transfected into HEK293T cells, and cells were grown 5 days. FcRn heterodimer was purified from supernatant using an IgG affinity column made by conjugating the LS bevacizumab variant to activated CH Sepharose beads (GE Healthcare) using standard NHS chemistry. Receptor was bound in PBS at pH 6.0, followed by elution in PBS at pH 7.4. Antibody and receptor concentrations were determined by bicinchoninic acid (BCA) assay (Pierce).

Antibodies were screened for binding to human FcRn at pH 6.0 using Affinity to FcRn was measured with an antigen-mediated antibody capture/human FcRn analyte format using a Biacore 3000 instrument (Biacore). VEGF in 10 mM sodium acetate, pH 4.5 buffer (Biacore) at 400 nM was immobilized to a CM5 chip (Biacore) to ˜3000 RUs using standard amine coupling. Anti-VEGF antibodies were immobilized on the VEGF surface to ˜400 RUs for higher affinity variants or ˜1200 RUs for IgG1 in pH 6.0 FcRn running buffer (50 mM Phosphate, pH 6.0, 150 mM NaCl, 0.005% Biacore surfactant P20). Analyte FcRn was diluted in FcRn running buffer at 2-fold serial dilutions and injected at 30 ul/min for 2 min followed by disassociation for 2 min. Starting concentration for native IgG1 was 1 uM while higher affinity variants started at 500 nM or less. Following background/drift subtraction and axis-zeroing, sensograms were fit globally to a 1:1 Langmuir binding model using the BIAevaluation software (Biacore).

Dissocation at pH 7.4 was evaluated using a more avid Biacore format in which FcRn was conjugated directly to the CM5 chip. Antibodies at 200 nM were bound in FcRn running buffer at pH 6.0, followed by dissociation at pH 6.0 in FcRn running buffer, followed by further dissociation at pH 7.4 in HBS-EP buffer (10 mM HEPES pH 7.4, 150 mM NaCl, 3 mM, EDTA, 0.005% v/v Surfactant P20, pH 7.4) (Biacore). Kinetic data at pH 7.4 were fit individually to a 1:1 Langmuir dissociation model to provide off-rate constants. This highly avid format provided stronger signal than the antigen capture method and so enabled pH 7.4 dissociation to be visualized for all of the antibodies. Consequently binding kinetics were nonstandard and fitted parameters reflected only relative dissociation and not true affinities.

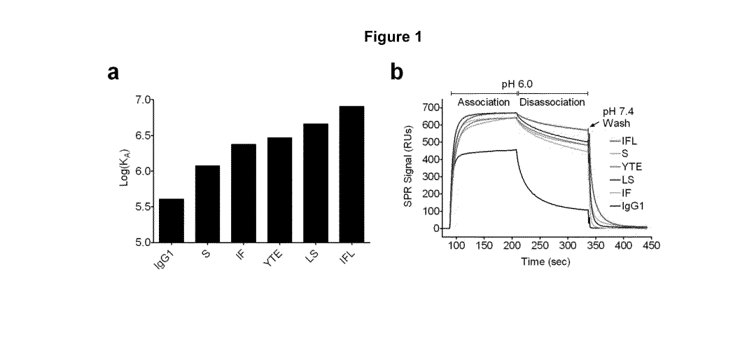

Biacore. FcRn binding data for select variants and native IgG1 are plotted in FIG. 1a, and equilibrium and kinetic binding constants are provided in Table 1. The binding experiment was carried out using an antigen-mediated antibody capture/human FcRn analyte format, and the data fit well to a 1:1 Langmuir binding model, suggesting that the values obtained represent true equilibrium constants. This is supported by agreement of our values for native IgG1 and the YTE variant with previously published data (see footnote to Table 1).

TABLE 1
Human FcRn binding parameters for engineered
bevacizumab Fc variants at pH 6.0

| | | kₒₙ | k_{off} | K_{D} | Fold |
|---|---|---|---|---|---|
| | Variant | (1/nM*s) | (1/s) | (nM) | K_{D} |
| IgG1 | Native IgG1 | 0.000121 | 0.297 | 2460^{a} | 1.00 |
| S | N434S | 0.000212 | 0.180 | 850 | 2.89 |
| IF | V259IA/308F | 0.000178 | 0.0752 | 424 | 5.80 |
| YTE | M252Y/S254T/T256E | 0.000162 | 0.0549 | 340^{b} | 7.24 |
| LS | M428L/N434S | 0.000255 | 0.0556 | 218 | 11.28 |
| IFL | V259IA/308F/M428L | 0.000192 | 0.0238 | 124 | 19.84 |

^{a}Literature values for binding of native IgG1 to FcRn are 2500 nM (Dall'Acqua W F et al., 2002, J Immunol 169: 5171-5180) and 2400 nM (Yeung Y A et al., 2009, J Immunol 182: 7663-7671).
^{b}Literature value for binding of YTE to FcRn is 230 nM (Dall'Acqua W F et al., 2002, J Immunol 169: 5171-5180).

These engineered variants provide between 3 and 20-fold greater binding to FcRn at pH 6.0 (FIG. 1a), with improvements due almost exclusively to slower off-rate (k_{off}) (Table 1). It has been suggested that an important parameter for such variants is low affinity at pH 7.4, based on the hypothesis that greater binding at serum pH would hinder recycled IgG release into the extracellular fluid and thus negatively impact half-life (Dall'Acqua W F et al., 2002, J Immunol 169:5171-5180; Datta-Mannan A et al., 2007, Drug metabolism and disposition: the biological fate of chemicals 35:86-94). We were unable to determine binding constants for all of the antibodies at pH 7.4 using the FcRn analyte format due to the rapid k_{off}. However, using a different format in which FcRn was conjugated directly to the Biacore chip (and antibody was analyte) we were able to increase avidity of the bound complex and thus obtain dissociation constants at pH 7.4 for all of the antibodies (FIG. 1b). Increased binding at pH 6.0 was accompanied by a proportional decrease in dissociation at pH 7.4: off-rate constants (k_{off}'s) of the antibodies were 1.6, 0.60, 0.33, 0.32, and 0.29, 0.13 s⁻¹ for IgG1, IF, LS, YTE, S, and IFL variants, respectively. Although these values do not represent true kinetic constants and are not comparable to the values in Table 1 due to the highly avid nature of the format, they nonetheless indicate a rapid dissociation at pH 7.4 (within seconds) for even the highest affinity variants.

All of the engineered variants maintained binding to antigen, protein A, and Fc gamma receptors (FcγR5) (data not shown). Variants showed comparable FcRn binding improvements in the context of an IgG2 isotype, as well as in antibodies that target other antigens (data not shown).

### Example 2

### Engineered Variants Extend Half-Life in hFcRn Mice

To test the half-life of engineered variants in vivo, PK experiments were performed in C57BL/6J (B6)-background mice that are homozygous for a knock-out allele of murine FcRn and heterozygous for a human FcRn transgene (mFcRn^{-/-}, hFcRn⁺) (Petkova S B et al., 2006, International immunology 18:1759-1769; Roopenian D C et al., 2003, J Immunol 170:3528-3533), referred to herein as hFcRn mice.

hFcRn mice for PK studies (mFcRn^{-/-} hFcRn Tg 276 heterozygote on a B6 background (Petkova S B et al., 2006, International Immunology 18:1759-1769) were produced by and obtained from The Jackson Laboratory. In-life portions of the hFcRn mouse PK studies were carried out at The Jackson Laboratory-West for anti-VEGF antibodies (Table 2, Studies M1 and M2), or at Xencor for anti-EGFR antibodies (Table 2, Study M3). Female mice were randomized by body weight into groups of 6 (M1 and M2) or 7 (M3) and given a single slow-push bolus tail vein injection of antibodies at 2 mg/kg. Blood (˜50 ul) was drawn from the orbital plexus using topical anesthetic at each time point, processed to serum, and stored at −80° C. until analysis. Study durations were 25-49 days.

All immunoassays were carried out at Xencor. Serum concentrations of anti-VEGF antibodies in hFcRn mouse PK studies M1 and M2 were detected using a general human immunoglobulin recognition format with DELFIA time resolved fluorescence (TRF) detection. Goat anti-human-Fc-specific polyclonal antibody (Jackson ImmunoResearch) was adsorbed to the plate surface, and bound analyte was reacted with europium-labelled goat anti-human IgG (PerkinElmer). An antigen-down immunoassay using DELFIA TRF detection was used to detect anti-EGFR antibody serum concentrations in hFcRn study M3. Recombinant EGFR (R&D Systems) was absorbed to the plate surface, and bound analyte was detected using europium-labelled goat anti-human kappa (IBL-America). For all assays, after blocking non-specific sites on the surface, the immobilized antibody was incubated with an appropriate dilution of samples, qualification standards, and serial dilution of calibration standards. Separate calibrator curves and quality control samples were made for each test article; during sample testing the calibrator curve and quality control sample set specific for each test article were used for the serum analysis. The amount of captured antibody was quantified by measurement of time-resolved fluorescence signal intensity and reduced with a 4-PL curve fit using SoftMax Pro (Molecular Devices).

PK parameters were determined for individual mice with a non-compartmental model using WinNonlin version 5.0.2 (Pharsight). Nominal timepoints and doses were used and all data points were equally weighted in the analysis. Mean serum concentration versus time profiles for each test article were fit with a 2-compartment model to generate the curve fit shown in the figures.

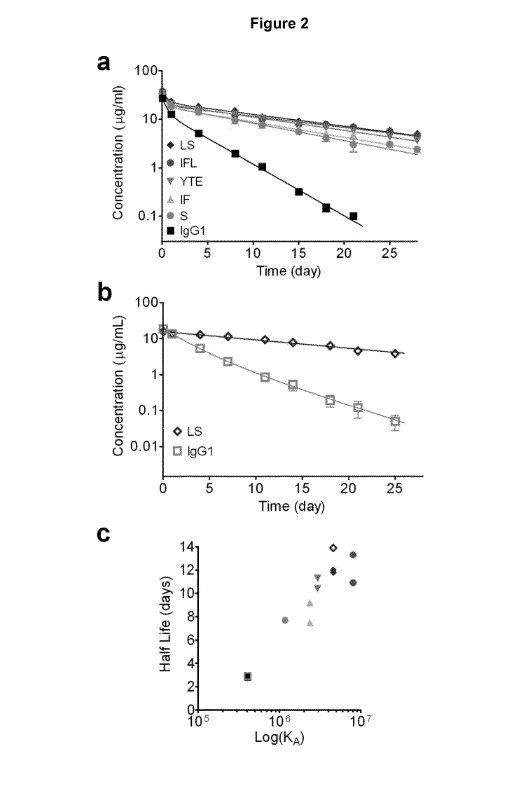

Serum concentration data for IgG1 anti-VEGF antibodies showed a dramatic improvement in half-life for the variants relative to native IgG1 (FIG. 2a). Fitted PK parameters from two separate studies, referred to as M1 and M2, indicated increases in β-phase half-life, the area under the concentration time curve (AUC), and the clearance of antibody from serum (Table 2). The best variants, M428L/N434S and V259I/V308F/M428L, extended half-life from approximately 3 to 13 days, providing between 4- and 5-fold improvement in serum half-life relative to native IgG1. The variants also demonstrated longer half-life in the context of the IgG2 isotype of bevacizumab in the hFcRn model, improving half-life from 5.9 days for native IgG2 to up to 16.5 days for the LS double variant (data not shown).

TABLE 2
PK parameters for hFcRn mouse and monkey studies

| | | | Animals | Half-Life^{c} | | | AUC^{c} | | Clearance^{c} | |
|---|---|---|---|---|---|---|---|---|---|---|
| | | | per | (day) | | | (day*ug/mL) | | (mL/day/kg) | |
| Antibody | Antigen^{a} | Study^{b} | Group | Mean | SD | Fold^{d} | Mean | SD | Mean | SD |
| IgG1 | VEGF | M1 | 6 | 2.8 | 0.3 | 1.0 | 69 | 10 | 29.4 | 4.6 |
| YTE | VEGF | M1 | 6 | 10.4 | 1.5 | 3.7 | 317 | 67 | 6.6 | 1.5 |
| S | VEGF | M1 | 6 | 7.7 | 1.5 | 2.8 | 228 | 75 | 10.0 | 4.6 |
| IF | VEGF | M1 | 6 | 9.2 | 1.5 | 3.3 | 262 | 47 | 7.9 | 1.4 |
| LS | VEGF | M1 | 6 | 12.0 | 2.9 | 4.3 | 400 | 112 | 5.5 | 2.0 |
| IFL | VEGF | M1 | 6 | 13.3 | 2.7 | 4.8 | 383 | 68 | 5.3 | 0.8 |
| IgG1 | VEGF | M2 | 6 | 2.9 | 0.4 | 1.0 | 73 | 6 | 27.6 | 2.3 |
| YTE | VEGF | M2 | 6 | 11.3 | 1.8 | 3.9 | 377 | 61 | 5.4 | 0.8 |
| IF | VEGF | M2 | 6 | 7.5 | 0.8 | 2.6 | 235 | 23 | 8.6 | 0.9 |
| LS | VEGF | M2 | 6 | 11.8 | 0.6 | 4.1 | 392 | 52 | 5.2 | 0.7 |
| IFL | VEGF | M2 | 6 | 10.9 | 0.6 | 3.8 | 295 | 55 | 7.0 | 1.3 |
| IgG1 | EGFR | M3 | 7 | 2.9 | 0.7 | 1.0 | 66 | 18 | 33.4 | 14 |
| LS | EGFR | M3 | 7 | 13.9 | 1.4 | 4.8 | 315 | 34 | 6.4 | 0.7 |
| IgG1 | VEGF | C1 | 2^{e} | 9.7 | | 1.0 | 823 | | 4.9 | |
| YTE | VEGF | C1 | 3 | 24.2 | 1.6 | 2.5 | 1919 | 210 | 2.1 | 0.2 |
| IF | VEGF | C1 | 3 | 16.2 | 6.4 | 1.7 | 1353 | 367 | 3.1 | 0.9 |
| LS | VEGF | C1 | 3 | 31.1 | 7.9 | 3.2 | 2661 | 791 | 1.6 | 0.6 |
| IFL | VEGF | C1 | 3 | 25.1 | 5.9 | 2.6 | 2302 | 923 | 1.9 | 0.8 |
| IgG1 | EGFR | C2 | 2^{e} | 1.5 | | 1.0 | 424 | | 18.5 | |
| LS | EGFR | C2 | 2 | 4.7 | | 3.1 | 1338 | | 5.7 | |

^{a}The Fv region of anti-VEGF antibodies was bevacizumab; the Fv region of anti-EGFR antibodies was C225 for the native IgG1 version or humanized cetuximab (huC225) for the LS Fc engineered version.
^{b}M refers to PK studies carried out in hFcRn mice, C refers to studies carried out in cynomolgous monkeys. Dose level and route: M1-M3 single i.v. bolus at 2 mg/kg, C1 single i.v. infusion at 4 mg/kg, C2 single i.v. infusion at 7.5 mg/kg.
^{c}Half-life, area under the curve (AUC), and clearance were computed for individual animals using noncompartment methods and are reported as the mean and standard deviation (SD).
^{d}Fold half-life = half-life (variant)/half-life (IgG1).
^{e}SD not calculated for N = 2 animals.

To evaluate the capacity of the variants to improve half-life in the context of antibodies targeting both circulating and cell surface antigens, the LS variant was constructed in a humanized version (huC225) of the anti-EGFR antibody cetuximab (C225) (Naramura M et al., 1993, Cancer Immunol Immunother 37:343-349) (Erbitux®, Imclone/Lilly), which is approved for the treatment of colorectal and head and neck cancers. The variant provided similar affinity improvement to human FcRn as for anti-VEGF, and binding to human EGFR antigen was unperturbed (data not shown). In hFcRn mice, the LS variant extended the half-life to 13.9 days relative to 2.9 days for cetuximab, resulting in an improvement of 5-fold (FIG. 2b, Table 2). The IgG1 version of huC225 also had a relatively short 2 day half-life (data not shown). Although these variable regions do not cross-react with murine EGFR, these results demonstrated broad applicability of the variants and gave us confidence in anti-EGFR as a test system for studying the impact of antigen sink in non-human primates.

Across the two anti-VEGF and one anti-EGFR hFcRn PK studies, a strong correlation was observed between antibody half-life and FcRn affinity at pH 6.0 (FIG. 2c). Moreover, the PK results for individual variants and native IgG1 were consistent and reproducible between the three studies (FIG. 2c). Together with the support provided by the monkey studies described below, these results further establish the hFcRn transgenic mouse as a model system for studying the relative PK properties of human IgG antibodies.

### Example 3

### Engineered Variants Extend Half-Life in Non-Human Primates

The PK properties of biologics in monkeys are well-established to be predictive of their properties in humans. A PK study was carried out in cynomolgus monkeys (macaca fascicularis) in order to evaluate the capacity of the variants to improve serum half-life in monkeys.

In-life portions were conducted at SNBL USA, LTD. All studies were approved by the SNBL IACUC, all test articles were well tolerated, and the animals were returned to colony stock upon study completion. For the anti-VEGF study, male cynomolgus monkeys (macaca fascicularis) weighing 2.3-5.1 kg were randomized by weight and divided into 5 groups of 3 monkeys/group. Monkeys were given a single, 1 hour intravenous infusion at 4 mg/kg in a dose volume of 10 mL/kg. One animal infused with bevacizumab died due to a procedural error 72 hour after drug infusion, this event was considered unrelated to test article. Consequently, serum concentration results are not reported for this animal. Blood samples (1 ml) were drawn from 5 minutes to 90 days after completion of the infusion, processed to serum and stored at −70° C. The anti-EGFR study was run similarly except that 2 groups of 2 monkeys/group were used (3 male and 1 female), the dose was given as a 30 minute intravenous infusion at 7.5 mg/kg in a dose volume of 7.5 mL/kg, and the study ran from 5 minutes to 21 days.

All immunoassays were carried out at Xencor. An antigen-down immunoassay using DELFIA TRF detection was used to detect anti-VEGF antibody serum concentrations in monkey study C1 and anti-EGFR antibody serum concentrations in monkey study C2. Recombinant EGFR(R&D Systems) or VEGF (PeproTech) was absorbed to the plate surface, and bound analyte was detected using europium-labelled goat anti-human kappa (IBL-America). For all assays, after blocking non-specific sites on the surface, the immobilized antibody was incubated with an appropriate dilution of samples, qualification standards, and serial dilution of calibration standards. Separate calibrator curves and quality control samples were made for each test article; during sample testing the calibrator curve and quality control sample set specific for each test article were used for the serum analysis. The amount of captured antibody was quantified by measurement of time-resolved fluorescence signal intensity and reduced with a 4-PL curve fit using SoftMax Pro (Molecular Devices).

PK parameters were determined for individual monkeys with a non-compartmental model using WinNonlin version 5.0.2 (Pharsight). Nominal timepoints and doses were used and all data points were equally weighted in the analysis. Mean serum concentration versus time profiles for each test article were fit with a 2-compartment model to generate the curve fit shown in the figures.

Binding improvements of the variants to cynomolgus FcRn at pH 6.0 were comparable to improvements for human FcRn, and the rank order of the variants in FcRn affinity was the same (data not shown). These results are not surprising given the high sequence homology of human and cynomolgus receptors (FcRn α-chain 98%, β2-microglobulin 91%).

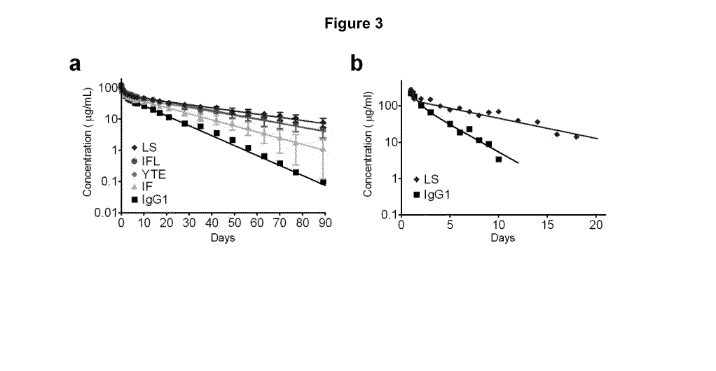

Three monkeys per group were injected intravenously (i.v.) with 4 mg/kg variant or native IgG1 anti-VEGF antibody. One of the monkeys in the native IgG1 group showed a drop in serum concentration early in the study, presumably due to immune-mediated clearance; serum concentration data were acquired to the full 90 days for all other monkeys. The results showed a large improvement in half-life for the variants relative to native IgG1 (FIG. 3a), consistent with the results obtained in hFcRn mice. Fitted parameters (Table 2) indicated increases in β-phase half-life, AUC, and the clearance of antibody from serum. The observed 9.7 day half-life for native IgG1 bevacizumab agrees with the published value (9.3 days) for a slightly lower (2 mg/kg) dose (Lin Y S et al., 1999, The Journal of Pharmacology and Experimental Therapeutics 288:371-378). Among the engineered antibodies, the LS double variant performed best, extending half-life from 9.7 to 31.1 days, a 3.2-fold improvement in serum half-life relative to native IgG1. These PK results obtained in monkeys are consistent with those obtained in hFcRn mice, validating the latter as a model system for assessing the in vivo PK properties of the variants, and supporting the conclusions from those studies.

A separate PK study in monkeys was carried out with anti-EGFR antibodies to assess half-life in the context of an antibody whose clearance is mediated by surface antigen (Lammerts van Bueren J J et al., 2006, Cancer research 66:7630-7638; Fan Z et al., 1994, The Journal of Biological Chemistry 269:27595-27602). Cetuximab and humanized cetuximab cross-react with cynomolgus EGFR (data not shown). The 7.5 mg/kg dose chosen for this study is in a range where the dose-clearance relationship is nonlinear. In our hands cetuximab had a half-life of 1.5 days (FIG. 3b, Table 2). Consistent with the bevacizumab results, the LS double variant anti-EGFR extended half-life to 4.7 days, reflecting a 3.1-fold improvement (FIG. 3b, Table 2).

### Example 4

### Improved Half-Life Results in Enhanced Efficacy for Anti-VEGF and -EGFR Antibodies

We wished to test whether the slower clearance of our PK-engineered antibodies resulted in improved exposure-related pharmacology. We therefore developed an hFcRn transgenic, Rag1^{-/-} immunodeficient mouse strain to enable the development of tumor models for both VEGF and EGFR systems in mice expressing human FcRn.

hFcRn/Rag1^{-/-} mice for xenograft studies (mFcRn^{-/-} hFcRn Tg 276 heterozygote Rag1^{-/-}) on a B6 background were produced at The Jackson Laboratory from an F₁ cross of mFcRn^{-/-} hFcRn Tg 276 homozygotes to mFcRn^{-/-} B6 Rag1^{-/-} mice, followed by selection of mFcRn^{-/-} hFcRn Tg 276 heterozygote Rag1^{-/-} mice in the F₂ generation. Human ovarian carcinoma SKOV-3 cells (ATCC) were cultured in McCoy's 5a medium (Invitrogen) with 10% fetal bovine serum (FBS). 5×10⁶ SKOV-3 cells were injected subcutaneously and mice bearing tumors of 25-60 mm³ (day 35) were selected for the study. Human epidermoid carcinoma A431 cells (ATCC) were cultured in RPMI 1640 medium (Mediatech) with 10% FBS. 10⁶ A431 cells were injected subcutaneously and mice bearing tumors of 20-122 mm3 (day 10) were selected for the study. Tumor-bearing mice were dosed intraperitoneally with PBS or 5 mg/kg antibody (native IgG1 or variant) once every 10 days (8-9 mice per group). Tumor volume was measured 1-2× per week using calibrated vernier calipers. All xenograft experimental procedures were approved by the respective Institutional Animal Care and Use Committees (IACUCs) and conducted in a manner to avoid or minimize distress or pain to animals.

An antigen-down immunoassay using DELFIA TRF detection was used to detect anti-VEGF antibody serum concentrations in the hFcRn/Rag1^{-/-} SKOV-3 xenograft study, and anti-EGFR antibody serum concentrations in the hFcRn/Rag1^{-/-} A431 xenograft study, as described above. PK parameters were determined for individual mice with a non-compartmental model as described above.

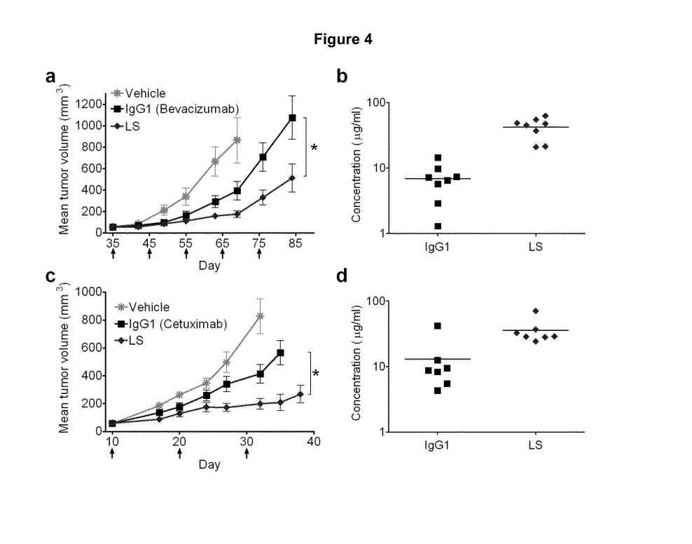

For VEGF, SKOV-3 tumors were established to 25-60 mm³ and then treated with either vehicle or 5 mg/kg native IgG1 or LS variant bevacizumab every 10 days. This dosing schedule approximated the half-life of the variant, but was 3-4 half-lives longer than the clearance rate of the native IgG1 version (Table 2). A statistically greater level of tumor reduction (p=0.028 at study termination) was observed for LS variant relative to the native IgG1 version (FIG. 4a). Consistent with the PK results in hFcRn mice (FIG. 3a), the variants reduced clearance in the hFcRn/Rag1^{-/-} mice (FIG. 4b), demonstrating the inverse correlation between tumor volume and serum concentration of antibody at study termination. A similar study in hFcRn/Rag1^{-/-} mice using the anti-EGFR antibodies showed similar improvements in tumor reduction (p=0.005) against established A431 epidermoid carcinoma tumors (FIG. 4c, d). These results indicate that the slower clearance of the variant antibodies leads to higher drug exposure and consequently greater tumor cytotoxicity.

### Example 5

### Immunoglobulin Constant Chains that Provide Extended Half-Life

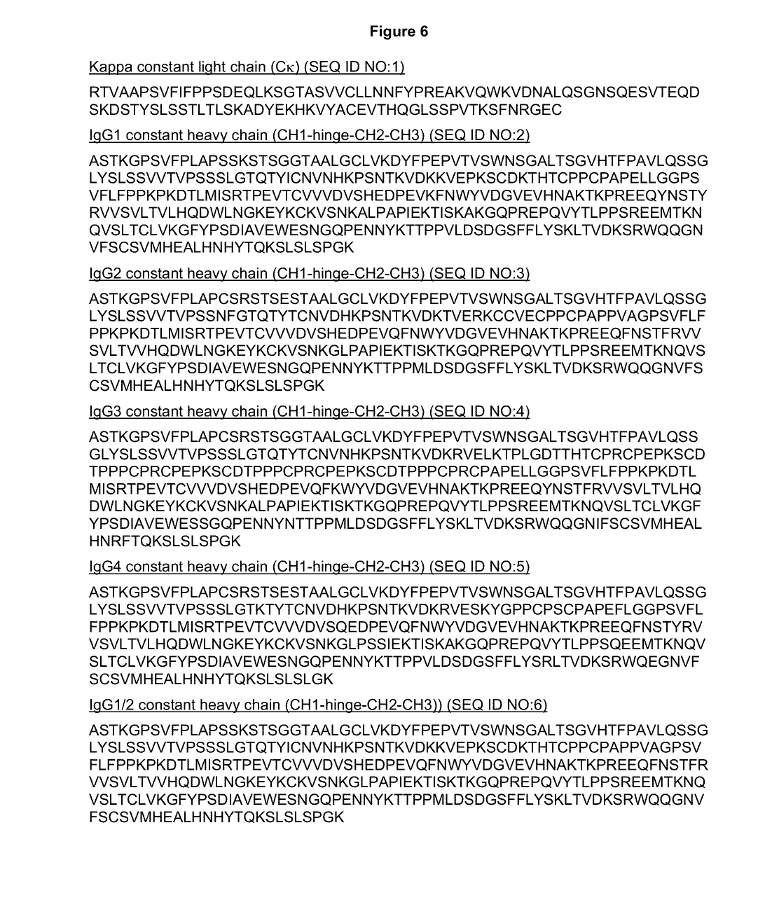

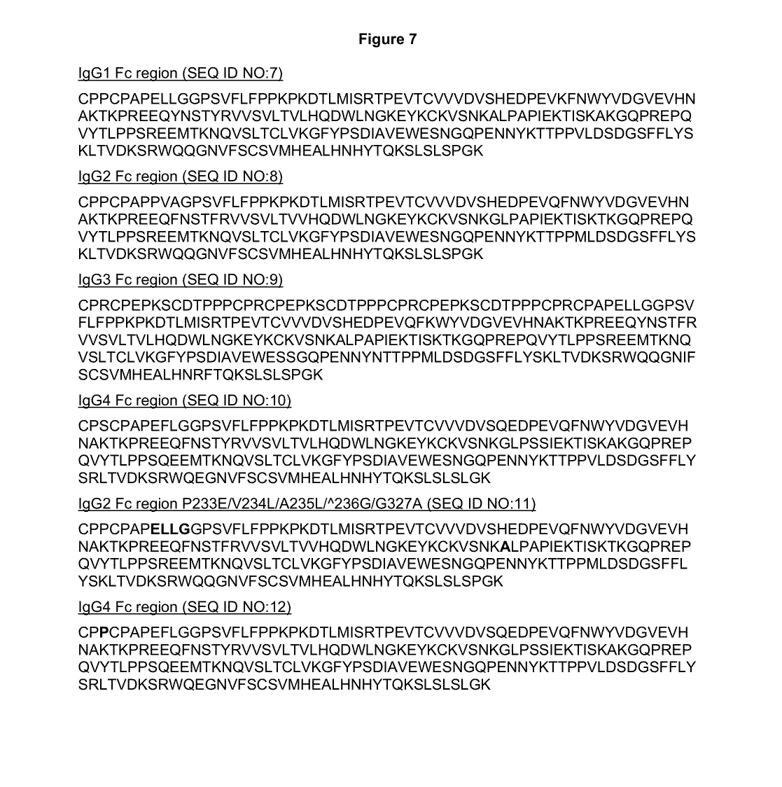

Amino acid sequences of exemplary parent constant regions are provided in FIG. 6. Amino acid sequences of exemplary parent Fc regions are provided in FIG. 7. As is well known in the art, isotypic substitutions (as illustrated in FIG. 5) can be made into these Fc regions to alter their properties. For example, the amino acid modifications P233E, V234L, A235L, the insertion ̂236G, and the substitution G327A can be incorporated into IgG2 to increase its effector function. As another example, the heavy chain exchange properties of IgG4 can be reduced by making the substitution S228P

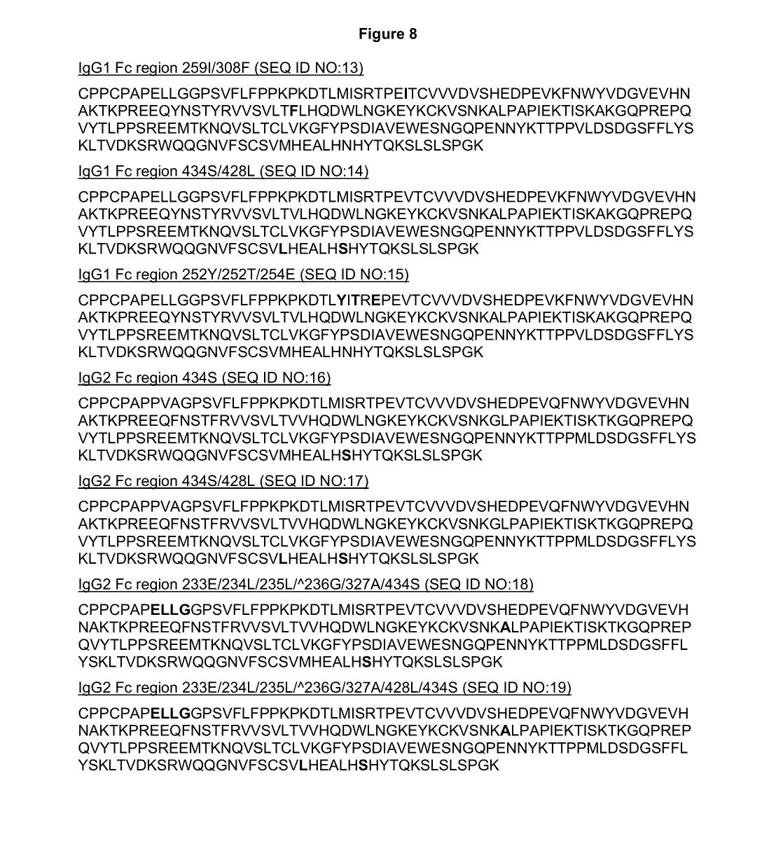

Amino acid sequences of variant Fc regions are provided in FIG. 8.

### Example 6

### Antibodies and Fc Fusions with Extended Half-Life

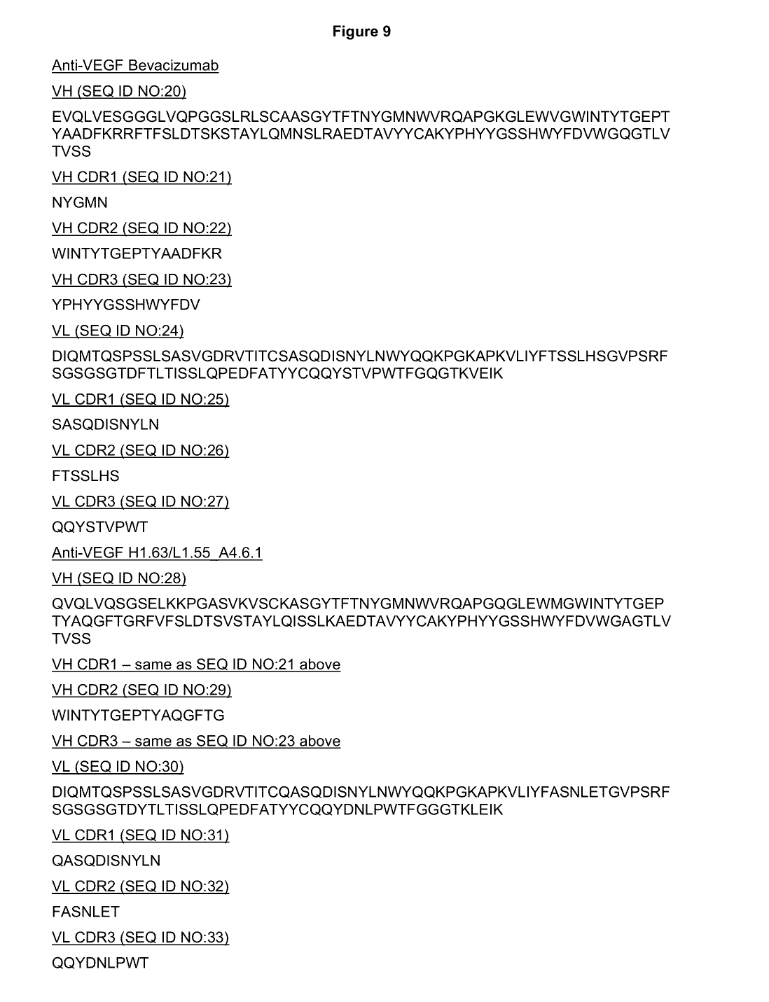

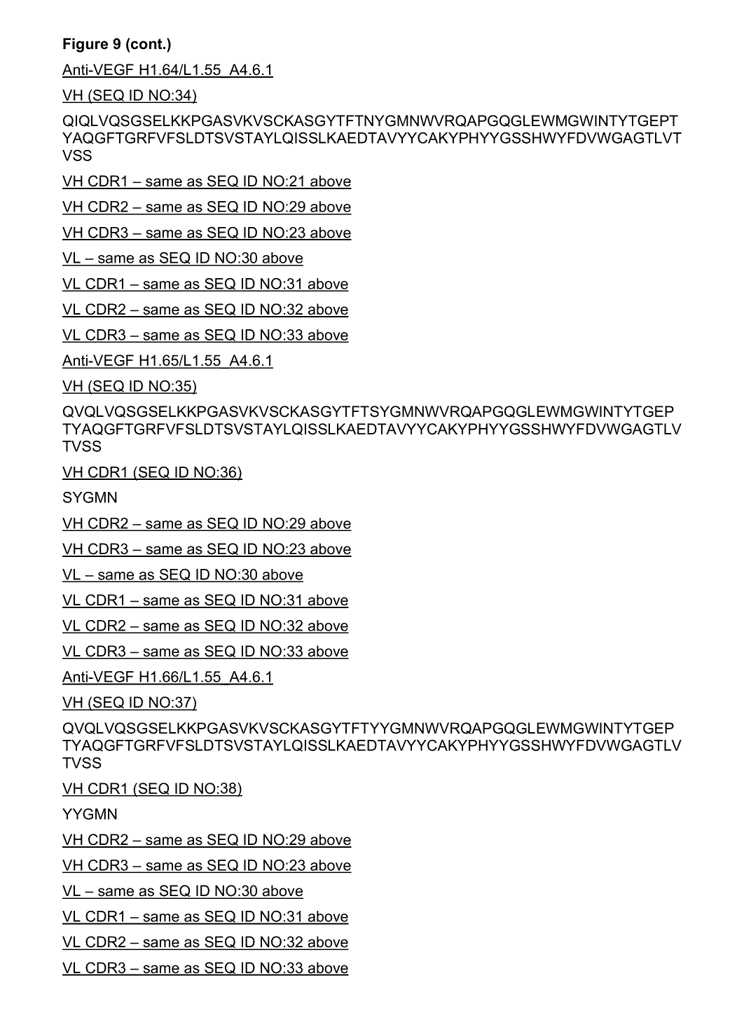

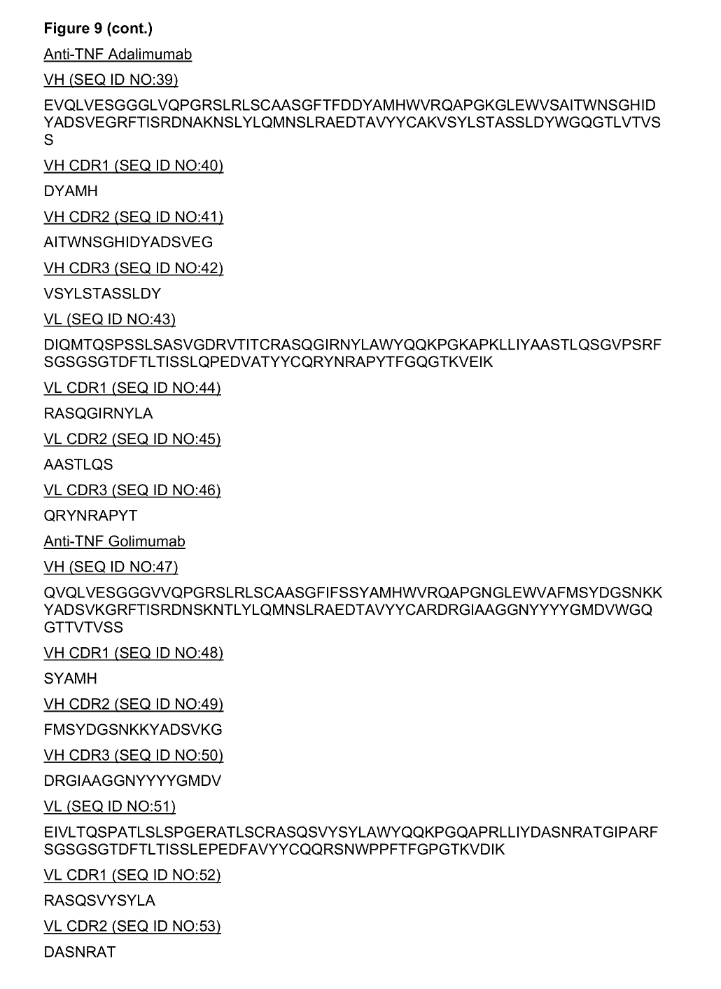

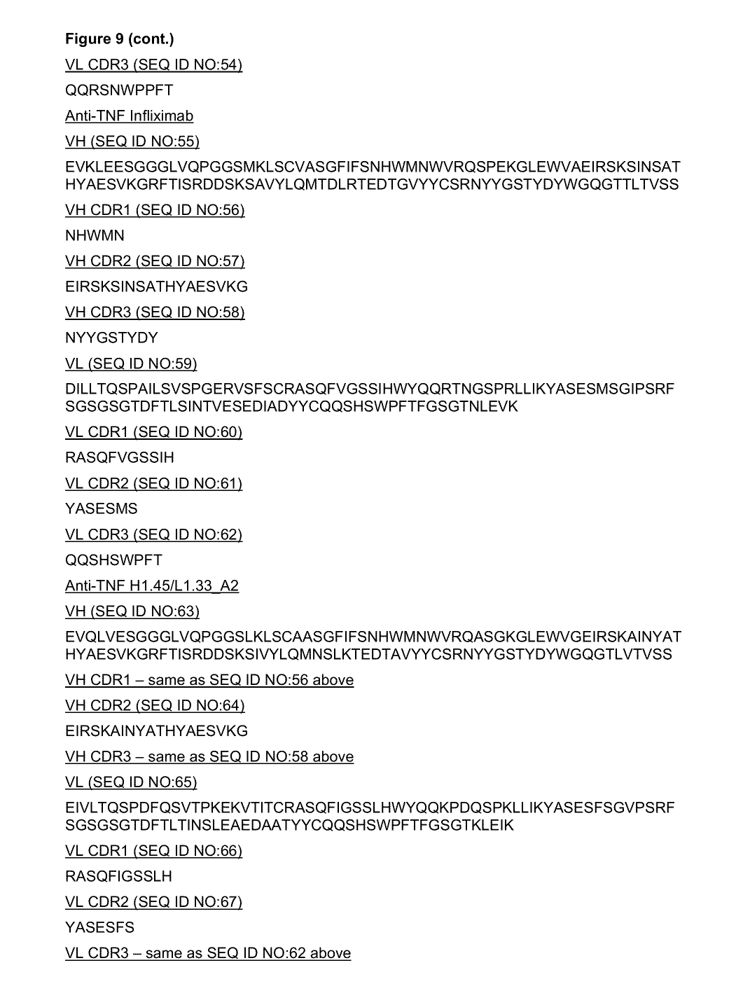

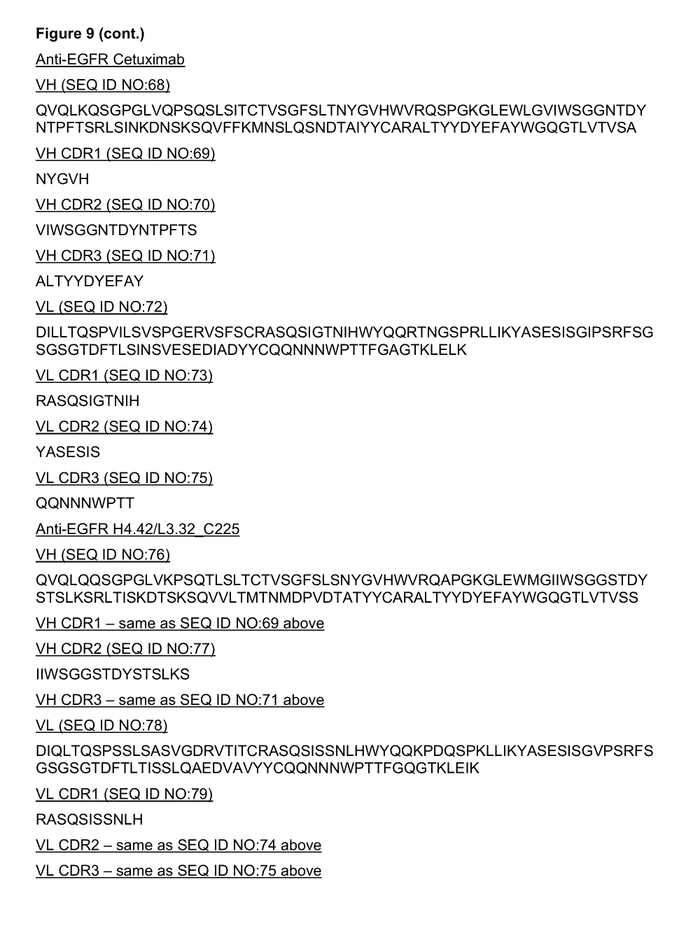

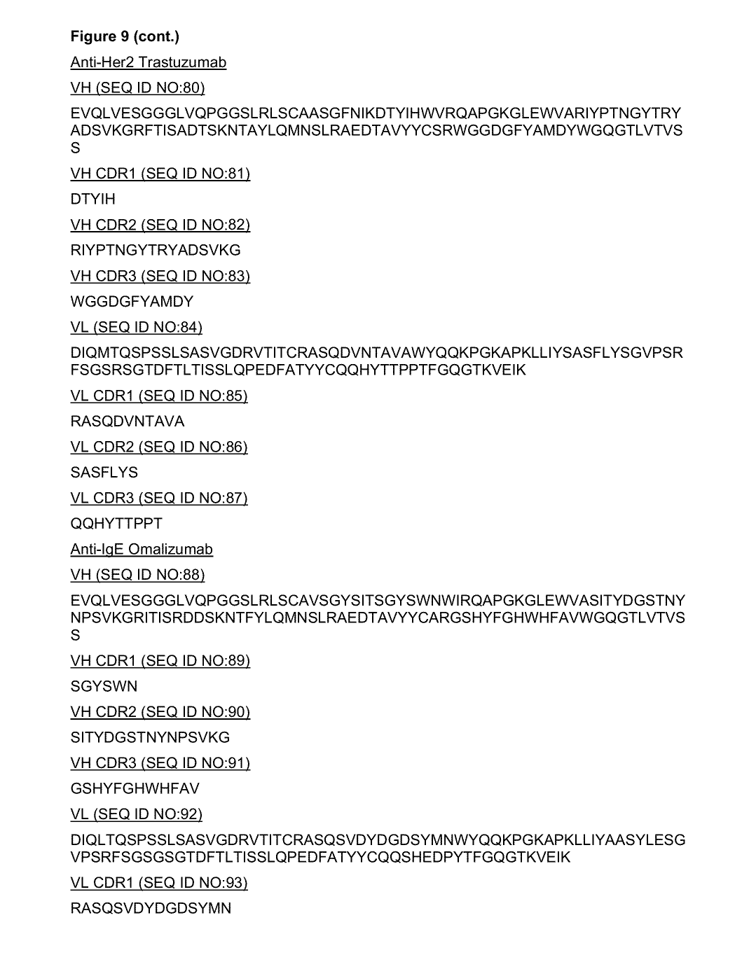

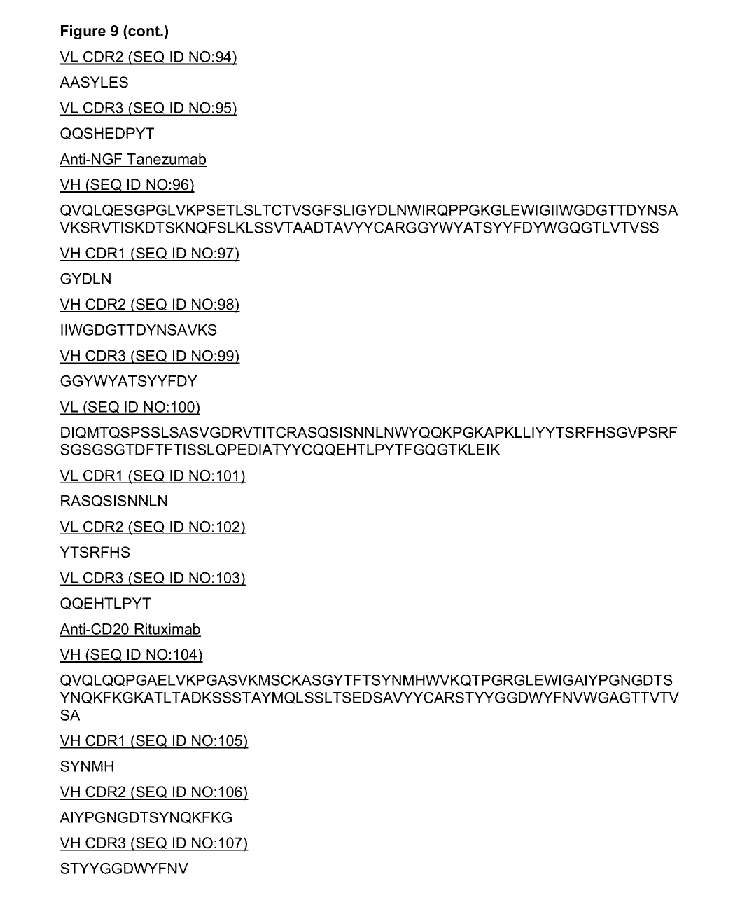

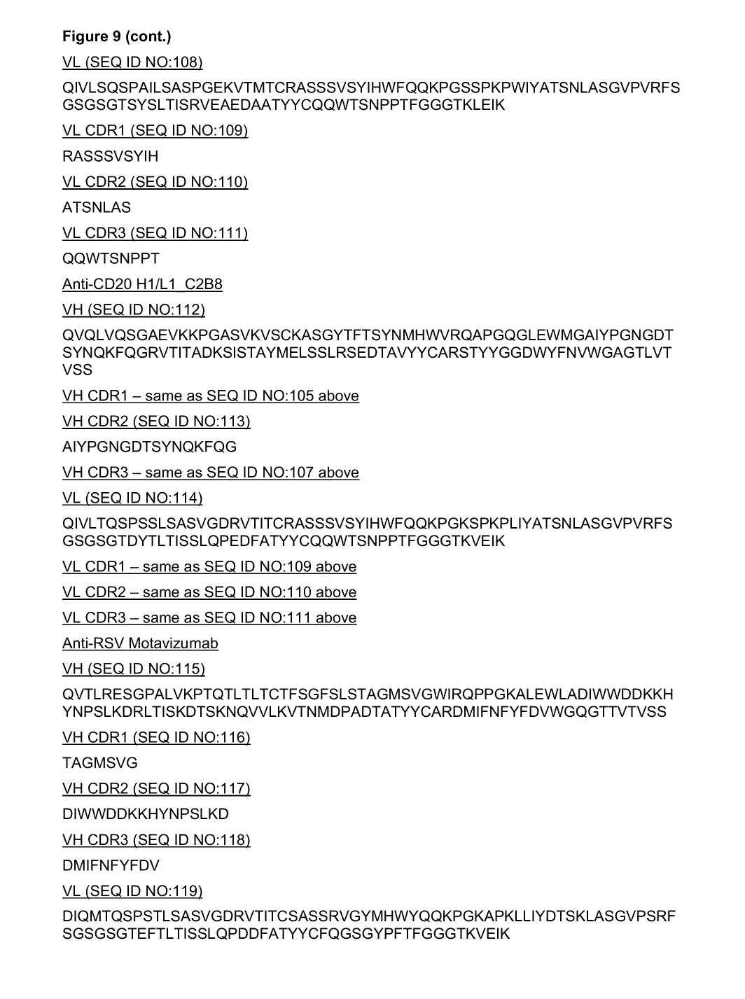

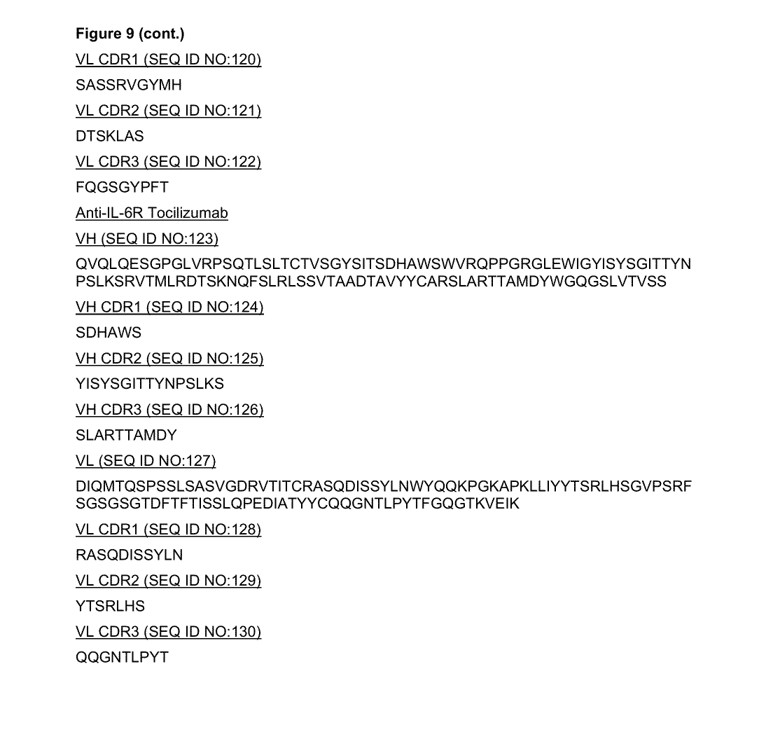

FIG. 9 provides amino acid sequences of the variable heavy (VH) and light (VL) regions, as well as the CDRs of these variable regions, of exemplary antibodies whose Fc region is modified to extend in vivo half-life. These exemplary antibodies include the anti-VEGF antibodies bevacizumab, H1.63/L1.55_A4.6.1, H1.64/L1.55_A4.6.1, H1.65/L1.55_A4.6.1, H1.66/L1.55_A4.6.1, the anti-TNF antibodies Adalimumab, Golimumab, Infliximab, and H1.45/L1.33_A2, the anti-EGFR antibodies Cetuximab and H4.42/L3.32_C225, the anti-Her2 antibody Trastuzumab, the anti-IgE antibody Omalizumab, the anti-NGF antibody Tanezumab, the anti-CD20 antibodies Rituximab and H1/L1_C2B8, the anti-RSV antibody Motavizumab, and the anti-IL-6R Tocilizumab.

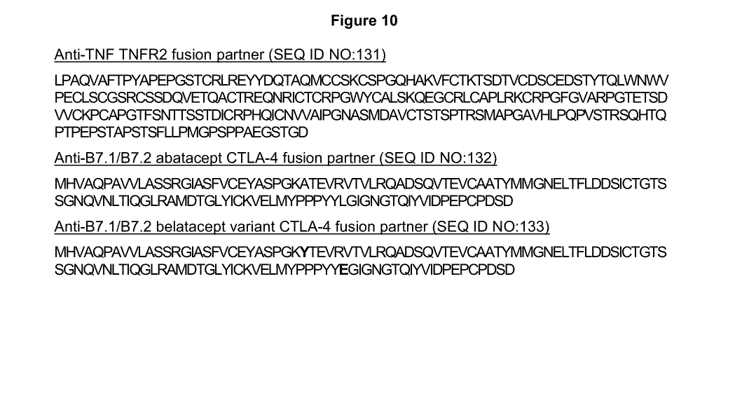

FIG. 10 provides amino acid sequences of Fc fusion partners that may be linked to a modified Fc region to extend in vivo half-life. Exemplary immunoadhesins include anti-TNF Fc fusions that comprise modified Fc regions linked to the receptor TNFR2, and anti-B7.1(CD80)/B7.2(CD86) Fc fusions that comprise modified Fc regions linked to Cytotoxic T-Lymphocyte Antigen 4 (CTLA-4) or variant versions of CTLA-4.

### Example 7

### Anti-TNF Immunoglobulins with Extended Half-Life

Optimized anti-TNF (TNFalpha, TNFα) antibodies were constructed by constructing a 428L/434S variant version of the antibody with adalimumab (Humira®), currently approved for the treatment of rheumatoid arthritis (RA), juvenile idiopathic arthritis (JIA), psoriatic arthritis (PsA), ankylosing spondylitis (AS), and Crohn's disease (CD). The amino acid sequences of the variable region and CDRs of this antibody are provided in FIG. 9. WT and variant antibodies were constructed, expressed, and purified as described above. Antibodies were tested for binding to human FcRn at pH 6.0 by Biacore. For measuring TNF binding, a CM4 chip was used to couple antibodies directly to the chip surface. EDC/NHS mix was diluted 2-fold, and used for activation for only 30 sec. All antibodies were diluted in pH 4 acetate buffer to 100 nM and coupled at 2 ul/min for 10 minutes followed by blocking with ethanolamine for 4 min. The RUs obtained were 380, 360, and 580 respectively. FC2 was coupled to Humira (Commercial), FC3 to XP_{—}6401, and FC4 to XP_{—}6755. recombinant human TNF was diluted in HBS-Ep (pH 7.4, Biacore) to 200, 100, 50, 25, 12.5, 6.25 and 0 nM and injected through all channels where FC1 served as background subtraction channel. Human TNF injection was at 30 ul/min for 2 min ON and 5 min OFF. For measurement of binding to human FcRn, a CM5 Biacore chip previously coupled to anti-hFab antibody is used. The running buffer for FcRn binding is pH 6.0 PBS. Each antibody was immobilized manually first by injecting 100 nM solution at 10 ul/min for appropriate duration to obtain RUs of ˜700 for WT-IgG1 or ˜400 for the variant. Then an automated kinjection method was started for a series of concentrations of the hFcRn. Due to fast off rate (disassociation) no regeneration was required for multiple FcRn injections.

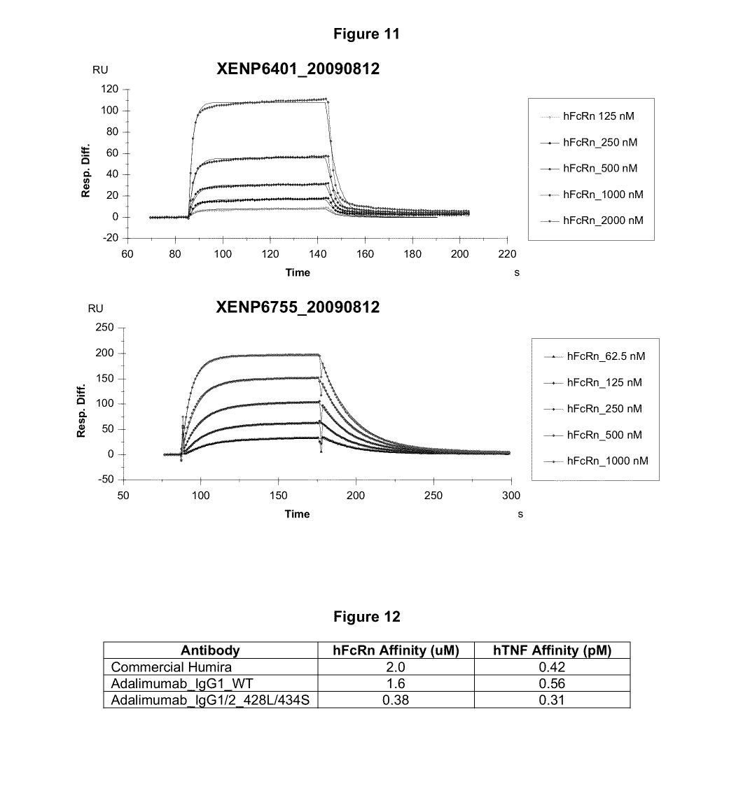

For each antibody, resulting sensograms were first processed by zeroing y-axis of all curves and finally subtracting the 0 nM trace from all other curves in the group. Resulting “Y-axis zeroed” and “buffer alone subtracted” curves were fitted with 1:1 langmuir group fit where RI was set to zero and Rmax was allowed to vary (TNF) or not (FcRn). FIG. 11 shows Biacore sensorgrams for binding of variant (XENP6401) and native IgG1 (XENP6755) versions of adalimumab to human FcRn. FIG. 12 shows affinities for binding of anti-TNF antibodies to human FcRn and human TNF as determined by Biacore. As can be seen, the variants improve FcRn affinity in the context of the anti-TNF antibody.

Whereas particular embodiments of the invention have been described above for purposes of illustration, it will be appreciated by those skilled in the art that numerous variations of the details may be made without departing from the invention as described in the appended claims. All references cited herein are incorporated in their entirety.

1-6. (canceled)

7. An antibody comprising:
a) a variable heavy chain domain comprising a vhCDR1 having SEQ ID NO:69, a vhCDR2 having SEQ ID NO:77 and a vhCDR3 having SEQ ID NO:71; and
b) a variable light chain domain comprising a vlCDR1 having SEQ ID NO:79, a vlCDR2 having SEQ ID NO:74 and a vlCDR3 having SEQ ID NO:75.

8. An antibody according to claim 7 wherein said variable heavy chain comprising SEQ ID NO:76 and said variable light chain comprising SEQ ID NO:78.

9. An antibody according to claim 8 wherein the Fc domain of said antibody has an amino acid sequence selected from the group consisting of SEQ ID NOs:13 to 19.

10. An antibody according to claim 8 wherein the Fc domain of said antibody has an amino acid sequence selected from the group consisting of SEQ ID NOs:7 to 12.

11. A composition comprising:
a) a first nucleic acid encoding a variable heavy chain according to claim 7; and
b) a second nucleic acid encoding a variable light chain according to claim 7.

12. A host cell comprising the composition of claim 11.

13. An expression vector comprising the composition of claim 11.

14. A method of treating a patient in need thereof with an antibody according to claim 7.


SEQUENCE LISTING (135 sequences)

SEQ ID NO: 1 - protein, 107 aa, Artificial Sequence [sequence omitted]

SEQ ID NO: 2 - protein, 330 aa, Artificial Sequence
  IgG1 constant heavy chain (CH1-hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 326 aa, Artificial Sequence
  IgG2 constant heavy chain (CH1-hinge-CH2-CH3)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 377 aa, Artificial Sequence
  IgG3 constant heavy chain (CH1-hinge-CH2-CH3)
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 327 aa, Artificial Sequence
  IgG4 constant heavy chain (CH1-hinge-CH2-CH3)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 329 aa, Artificial Sequence
  IgG1/2 constant heavy chain (CH1-hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 222 aa, Artificial Sequence
  IgG1 Fc region
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 221 aa, Artificial Sequence
  IgG2 Fc region
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 267 aa, Artificial Sequence
  IgG3 Fc region
CPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 10 - protein, 222 aa, Artificial Sequence
  IgG4 Fc region
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 222 aa, Artificial Sequence
  IgG2 Fc region P233E/V234L/A235L/{circumflex over ())}{circumflex over (})}236G/G327A
CPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKALPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 222 aa, Artificial Sequence
  IgG4 Fc region
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 222 aa, Artificial Sequence
  IgG1 Fc region 259I/308F
CPPCPAPELLGGPSVFLFPPKPKDTLMISRTPEITCVVVDVSHEDPEVKFNWYVDGVEVHNAKTKPREEQYNSTYRVVSVLTFLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 222 aa, Artificial Sequence
  IgG1 Fc region 434S/428L
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 15 - protein, 222 aa, Artificial Sequence
  IgG1 Fc region 252Y/252T/254E
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 221 aa, Artificial Sequence
  IgG2 Fc region 434S
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPGK

SEQ ID NO: 17 - protein, 221 aa, Artificial Sequence
  IgG2 Fc region 434S/428L
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 18 - protein, 222 aa, Artificial Sequence
  IgG2 Fc region 233E/234L/235L/{circumflex over ())}{circumflex over (})}236G/327A/434S
CPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKALPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPGK

SEQ ID NO: 19 - protein, 222 aa, Artificial Sequence
  IgG2 Fc region 233E/234L/235L/{circumflex over ())}{circumflex over (})}236G/327A/428L/434S
CPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKALPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 20 - protein, 123 aa, Artificial Sequence [sequence omitted]

SEQ ID NO: 21 - protein, 5 aa, Artificial Sequence
  Anti-VEGF Bevacizumab VH CDR1 variable region
NYGMN

SEQ ID NO: 22 - protein, 17 aa, Artificial Sequence
  Anti-VEGF Bevacizumab VH CDR2 variable region
WINTYTGEPTYAADFKR

SEQ ID NO: 23 - protein, 14 aa, Artificial Sequence
  Anti-VEGF Bevacizumab VH CDR3 variable region
YPHYYGSSHWYFDV

SEQ ID NO: 24 - protein, 107 aa, Artificial Sequence [sequence omitted]

SEQ ID NO: 25 - protein, 11 aa, Artificial Sequence
  Anti-VEGF Bevacizumab VL CDR1 variable region
SASQDISNYLN

SEQ ID NO: 26 - protein, 7 aa, Artificial Sequence
  Anti-VEGF Bevacizumab VL CDR2 variable region
FTSSLHS

SEQ ID NO: 27 - protein, 9 aa, Artificial Sequence
  Anti-VEGF Bevacizumab VL CDR3 variable region
QQYSTVPWT

SEQ ID NO: 28 - protein, 123 aa, Artificial Sequence
  Anti-VEGF H1.63/L1.55_A4.6.1 VH variable region
QVQLVQSGSELKKPGASVKVSCKASGYTFTNYGMNWVRQAPGQGLEWMGWINTYTGEPTYAQGFTGRFVFSLDTSVSTAYLQISSLKAEDTAVYYCAKYPHYYGSSHWYFDVWGAGTLVTVSS

SEQ ID NO: 29 - protein, 17 aa, Artificial Sequence
  Anti-VEGF H1.63/L1.55_A4.6.1 VH CDR2 variable region
WINTYTGEPTYAQGFTG

SEQ ID NO: 30 - protein, 107 aa, Artificial Sequence
  Anti-VEGF H1.63/L1.55_A4.6.1 VL variable region
DIQMTQSPSSLSASVGDRVTITCQASQDISNYLNWYQQKPGKAPKVLIYFASNLETGVPSRFSGSGSGTDYTLTISSLQPEDFATYYCQQYDNLPWTFGGGTKLEIK

SEQ ID NO: 31 - protein, 11 aa, Artificial Sequence
  Anti-VEGF H1.63/L1.55_A4.6.1 VL CDR1 variable region
QASQDISNYLN

SEQ ID NO: 32 - protein, 7 aa, Artificial Sequence
  Anti-VEGF H1.63/L1.55_A4.6.1 VL CDR2 variable region
FASNLET

SEQ ID NO: 33 - protein, 9 aa, Artificial Sequence
  Anti-VEGF H1.63/L1.55_A4.6.1 VL CDR3 variable region
QQYDNLPWT

SEQ ID NO: 34 - protein, 123 aa, Artificial Sequence
  Anti-VEGF H1.64/L1.55_A4.6.1 VH variable region
QIQLVQSGSELKKPGASVKVSCKASGYTFTNYGMNWVRQAPGQGLEWMGWINTYTGEPTYAQGFTGRFVFSLDTSVSTAYLQISSLKAEDTAVYYCAKYPHYYGSSHWYFDVWGAGTLVTVSS

SEQ ID NO: 35 - protein, 123 aa, Artificial Sequence
  Anti-VEGF H1.65/L1.55_A4.6.1 VH variable region
QVQLVQSGSELKKPGASVKVSCKASGYTFTSYGMNWVRQAPGQGLEWMGWINTYTGEPTYAQGFTGRFVFSLDTSVSTAYLQISSLKAEDTAVYYCAKYPHYYGSSHWYFDVWGAGTLVTVSS

SEQ ID NO: 36 - protein, 5 aa, Artificial Sequence
  Anti-VEGF H1.65/L1.55_A4.6.1 VH CDR1 variable region
SYGMN

SEQ ID NO: 37 - protein, 123 aa, Artificial Sequence
  Anti-VEGF H1.66/L1.55_A4.6.1 VH variable region
QVQLVQSGSELKKPGASVKVSCKASGYTFTYYGMNWVRQAPGQGLEWMGWINTYTGEPTYAQGFTGRFVFSLDTSVSTAYLQISSLKAEDTAVYYCAKYPHYYGSSHWYFDVWGAGTLVTVSS

SEQ ID NO: 38 - protein, 5 aa, Artificial Sequence
  Anti-VEGF H1.66/L1.55_A4.6.1 VH CDR1 variable region
YYGMN

SEQ ID NO: 39 - protein, 121 aa, Artificial Sequence [sequence omitted]

SEQ ID NO: 40 - protein, 5 aa, Artificial Sequence
  Anti-TNF Adalimumab VH CDR1 variable region
DYAMH

SEQ ID NO: 41 - protein, 17 aa, Artificial Sequence
  Anti-TNF Adalimumab VH CDR2 variable region
AITWNSGHIDYADSVEG

SEQ ID NO: 42 - protein, 12 aa, Artificial Sequence
  Anti-TNF Adalimumab VH CDR3 variable region
VSYLSTASSLDY

SEQ ID NO: 43 - protein, 107 aa, Artificial Sequence [sequence omitted]

SEQ ID NO: 44 - protein, 11 aa, Artificial Sequence
  VL CDR1 variable region
RASQGIRNYLA

SEQ ID NO: 45 - protein, 7 aa, Artificial Sequence
  Anti-TNF Adalimumab VL CDR2 variable region
AASTLQS

SEQ ID NO: 46 - protein, 9 aa, Artificial Sequence
  Anti-TNF Adalimumab VL CDR3 variable region
QRYNRAPYT

SEQ ID NO: 47 - protein, 126 aa, Artificial Sequence
  Anti-TNF Golimumab VH variable region
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 48 - protein, 5 aa, Artificial Sequence
  Anti-TNF Golimumab VH CDR1 variable region
SYAMH

SEQ ID NO: 49 - protein, 17 aa, Artificial Sequence
  Anti-TNF Golimumab VH CDR2 variable region
FMSYDGSNKKYADSVKG

SEQ ID NO: 50 - protein, 17 aa, Artificial Sequence
  Anti-TNF Golimumab VH CDR3 variable region
DRGIAAGGNYYYYGMDV

SEQ ID NO: 51 - protein, 108 aa, Artificial Sequence
  Anti-TNF Golimumab VL variable region
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 52 - protein, 11 aa, Artificial Sequence
  Anti-TNF Golimumab VL CDR1 variable region
RASQSVYSYLA

SEQ ID NO: 53 - protein, 7 aa, Artificial Sequence
  Anti-TNF Golimumab VL CDR2 variable region
DASNRAT

SEQ ID NO: 54 - protein, 10 aa, Artificial Sequence
  Anti-TNF Golimumab VL CDR3 variable region
QQRSNWPPFT

SEQ ID NO: 55 - protein, 120 aa, Artificial Sequence
  Anti-TNF Infliximab VH variable region
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSS

SEQ ID NO: 56 - protein, 5 aa, Artificial Sequence
  Anti-TNF Infliximab VH CDR1 variable region
NHWMN

SEQ ID NO: 57 - protein, 19 aa, Artificial Sequence
  Anti-TNF Infliximab VH CDR2 variable region
EIRSKSINSATHYAESVKG

SEQ ID NO: 58 - protein, 9 aa, Artificial Sequence
  Anti-TNF Infliximab VH CDR3 variable region
NYYGSTYDY

SEQ ID NO: 59 - protein, 107 aa, Artificial Sequence
  Anti-TNF Infliximab VL variable region
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 60 - protein, 11 aa, Artificial Sequence
  Anti-TNF Infliximab VL CDR1 variable region
RASQFVGSSIH

SEQ ID NO: 61 - protein, 7 aa, Artificial Sequence
  Anti-TNF Infliximab VL CDR2 variable region
YASESMS

SEQ ID NO: 62 - protein, 9 aa, Artificial Sequence
  Anti-TNF Infliximab VL CDR3 variable region
QQSHSWPFT

SEQ ID NO: 63 - protein, 120 aa, Artificial Sequence
  Anti-TNF H1.45/L1.33_A2 VH variable region
EVQLVESGGGLVQPGGSLKLSCAASGFIFSNHWMNWVRQASGKGLEWVGEIRSKAINYATHYAESVKGRFTISRDDSKSIVYLQMNSLKTEDTAVYYCSRNYYGSTYDYWGQGTLVTVSS

SEQ ID NO: 64 - protein, 19 aa, Artificial Sequence
  Anti-TNF H1.45/L1.33_A2 VH CDR2 variable region
EIRSKAINYATHYAESVKG

SEQ ID NO: 65 - protein, 107 aa, Artificial Sequence
  Anti-TNF H1.45/L1.33_A2 VL variable region
EIVLTQSPDFQSVTPKEKVTITCRASQFIGSSLHWYQQKPDQSPKLLIKYASESFSGVPSRFSGSGSGTDFTLTINSLEAEDAATYYCQQSHSWPFTFGSGTKLEIK

SEQ ID NO: 66 - protein, 11 aa, Artificial Sequence
  Anti-TNF H1.45/L1.33_A2 VL CDR1 variable region
RASQFIGSSLH

SEQ ID NO: 67 - protein, 7 aa, Artificial Sequence
  Anti-TNF H1.45/L1.33_A2 VL CDR2 variable region
YASESFS

SEQ ID NO: 68 - protein, 119 aa, Artificial Sequence
  Anti-EGFR Cetuximab VH variable region
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 69 - protein, 5 aa, Artificial Sequence
  Anti-EGFR Cetuximab VH CDR1 variable region
NYGVH

SEQ ID NO: 70 - protein, 16 aa, Artificial Sequence
  Anti-EGFR Cetuximab VH CDR2 variable region
VIWSGGNTDYNTPFTS

SEQ ID NO: 71 - protein, 11 aa, Artificial Sequence
  Anti-EGFR Cetuximab VH CDR3 variable region
ALTYYDYEFAY

SEQ ID NO: 72 - protein, 107 aa, Artificial Sequence
  Anti-EGFR Cetuximab VL variable region
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 73 - protein, 11 aa, Artificial Sequence
  Anti-EGFR Cetuximab VL CDR1 variable region
RASQSIGTNIH

SEQ ID NO: 74 - protein, 7 aa, Artificial Sequence
  Anti-EGFR Cetuximab VL CDR2 variable region
YASESIS

SEQ ID NO: 75 - protein, 9 aa, Artificial Sequence
  Anti-EGFR Cetuximab VL CDR3 variable region
QQNNNWPTT

SEQ ID NO: 76 - protein, 119 aa, Artificial Sequence
  Anti-EGFR H4.42/L3.32_C225 VH variable region
QVQLQQSGPGLVKPSQTLSLTCTVSGFSLSNYGVHWVRQAPGKGLEWMGIIWSGGSTDYSTSLKSRLTISKDTSKSQVVLTMTNMDPVDTATYYCARALTYYDYEFAYWGQGTLVTVSS

SEQ ID NO: 77 - protein, 16 aa, Artificial Sequence
  Anti-EGFR H4.42/L3.32_C225 VH CDR2 variable region
IIWSGGSTDYSTSLKS

SEQ ID NO: 78 - protein, 107 aa, Artificial Sequence
  Anti-EGFR H4.42/L3.32_C225 VL variable region
DIQLTQSPSSLSASVGDRVTITCRASQSISSNLHWYQQKPDQSPKLLIKYASESISGVPSRFSGSGSGTDFTLTISSLQAEDVAVYYCQQNNNWPTTFGQGTKLEIK

SEQ ID NO: 79 - protein, 11 aa, Artificial Sequence
  Anti-EGFR H4.42/L3.32_C225 VL CDR1 variable region
RASQSISSNLH

SEQ ID NO: 80 - protein, 120 aa, Artificial Sequence [sequence omitted]

SEQ ID NO: 81 - protein, 5 aa, Artificial Sequence
  Anti-Her2 Trastuzumab VH CDR1 variable region
DTYIH

SEQ ID NO: 82 - protein, 17 aa, Artificial Sequence
  Anti-Her2 Trastuzumab VH CDR2 variable region
RIYPTNGYTRYADSVKG

SEQ ID NO: 83 - protein, 11 aa, Artificial Sequence
  Anti-Her2 Trastuzumab VH CDR3 variable region
WGGDGFYAMDY

SEQ ID NO: 84 - protein, 107 aa, Artificial Sequence [sequence omitted]

SEQ ID NO: 85 - protein, 11 aa, Artificial Sequence
  Anti-Her2 Trastuzumab VL CDR1 variable region
RASQDVNTAVA

SEQ ID NO: 86 - protein, 7 aa, Artificial Sequence
  Anti-Her2 Trastuzumab VL CDR2 variable region
SASFLYS

SEQ ID NO: 87 - protein, 9 aa, Artificial Sequence
  Anti-Her2 Trastuzumab VL CDR3 variable region
QQHYTTPPT

SEQ ID NO: 88 - protein, 121 aa, Artificial Sequence [sequence omitted]

SEQ ID NO: 89 - protein, 6 aa, Artificial Sequence
  Anti-IgE Omalizumab VH CDR1 variable region
SGYSWN

SEQ ID NO: 90 - protein, 16 aa, Artificial Sequence
  Anti-IgE Omalizumab VH CDR2 variable region
SITYDGSTNYNPSVKG

SEQ ID NO: 91 - protein, 12 aa, Artificial Sequence
  Anti-IgE Omalizumab VH CDR3 variable region
GSHYFGHWHFAV

SEQ ID NO: 92 - protein, 111 aa, Artificial Sequence [sequence omitted]

SEQ ID NO: 93 - protein, 15 aa, Artificial Sequence
  Anti-IgE Omalizumab VL CDR1 variable region
RASQSVDYDGDSYMN

SEQ ID NO: 94 - protein, 7 aa, Artificial Sequence
  Anti-IgE Omalizumab VL CDR2 variable region
AASYLES

SEQ ID NO: 95 - protein, 9 aa, Artificial Sequence
  Anti-IgE Omalizumab VL CDR3 variable region
QQSHEDPYT

SEQ ID NO: 96 - protein, 121 aa, Artificial Sequence [sequence omitted]

SEQ ID NO: 97 - protein, 5 aa, Artificial Sequence
  Anti-NGF Tanezumab VH CDR1 variable region
GYDLN

SEQ ID NO: 98 - protein, 16 aa, Artificial Sequence
  Anti-NGF Tanezumab VH CDR2 variable region
IIWGDGTTDYNSAVKS

SEQ ID NO: 99 - protein, 13 aa, Artificial Sequence
  Anti-NGF Tanezumab VH CDR3 variable region
GGYWYATSYYFDY

SEQ ID NO: 100 - protein, 107 aa, Artificial Sequence [sequence omitted]

SEQ ID NO: 101 - protein, 11 aa, Artificial Sequence
  Anti-NGF Tanezumab VL CDR1 variable region
RASQSISNNLN

SEQ ID NO: 102 - protein, 7 aa, Artificial Sequence
  Anti-NGF Tanezumab VL CDR2 variable region
YTSRFHS

SEQ ID NO: 103 - protein, 9 aa, Artificial Sequence
  Anti-NGF Tanezumab VL CDR3 variable region
QQEHTLPYT

SEQ ID NO: 104 - protein, 121 aa, Artificial Sequence [sequence omitted]

SEQ ID NO: 105 - protein, 5 aa, Artificial Sequence
  Anti-CD20 Rituximab VH CDR1 variable region
SYNMH

SEQ ID NO: 106 - protein, 17 aa, Artificial Sequence
  Anti-CD20 Rituximab VH CDR2 variable region
AIYPGNGDTSYNQKFKG

SEQ ID NO: 107 - protein, 12 aa, Artificial Sequence
  Anti-CD20 Rituximab VH CDR3 variable region
STYYGGDWYFNV

SEQ ID NO: 108 - protein, 106 aa, Artificial Sequence [sequence omitted]

SEQ ID NO: 109 - protein, 10 aa, Artificial Sequence
  Anti-CD20 Rituximab VL CDR1 variable region
RASSSVSYIH

SEQ ID NO: 110 - protein, 7 aa, Artificial Sequence
  Anti-CD20 Rituximab VL CDR2 variable region
ATSNLAS

SEQ ID NO: 111 - protein, 9 aa, Artificial Sequence
  Anti-CD20 Rituximab VL CDR3 variable region
QQWTSNPPT

SEQ ID NO: 112 - protein, 121 aa, Artificial Sequence
  Anti-CD20 H1/L1_C2B8 VH variable region
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSS

SEQ ID NO: 113 - protein, 17 aa, Artificial Sequence
  Anti-CD20 H1/L1_C2B8 VH CDR2 variable region
AIYPGNGDTSYNQKFQG

SEQ ID NO: 114 - protein, 106 aa, Artificial Sequence
  Anti-CD20 H1/L1_C2B8 VL variable region
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 115 - protein, 120 aa, Artificial Sequence [sequence omitted]

SEQ ID NO: 116 - protein, 7 aa, Artificial Sequence
  Anti-RSV Motavizumab VH CDR1 variable region
TAGMSVG

SEQ ID NO: 117 - protein, 16 aa, Artificial Sequence
  Anti-RSV Motavizumab VH CDR2 variable region
DIWWDDKKHYNPSLKD

SEQ ID NO: 118 - protein, 10 aa, Artificial Sequence
  Anti-RSV Motavizumab VH CDR3 variable region
DMIFNFYFDV

SEQ ID NO: 119 - protein, 106 aa, Artificial Sequence [sequence omitted]

SEQ ID NO: 120 - protein, 10 aa, Artificial Sequence
  Anti-RSV Motavizumab VL CDR1 variable region
SASSRVGYMH

SEQ ID NO: 121 - protein, 7 aa, Artificial Sequence
  Anti-RSV Motavizumab VL CDR2 variable region
DTSKLAS

SEQ ID NO: 122 - protein, 9 aa, Artificial Sequence
  Anti-RSV Motavizumab VL CDR3 variable region
FQGSGYPFT

SEQ ID NO: 123 - protein, 119 aa, Artificial Sequence [sequence omitted]

SEQ ID NO: 124 - protein, 6 aa, Artificial Sequence
  Anti-IL-6R Tocilizumab VH CDR1 variable region
SDHAWS

SEQ ID NO: 125 - protein, 16 aa, Artificial Sequence
  Anti-IL-6R Tocilizumab VH CDR2 variable region
YISYSGITTYNPSLKS

SEQ ID NO: 126 - protein, 10 aa, Artificial Sequence
  Anti-IL-6R Tocilizumab VH CDR3 variable region
SLARTTAMDY

SEQ ID NO: 127 - protein, 107 aa, Artificial Sequence [sequence omitted]

SEQ ID NO: 128 - protein, 11 aa, Artificial Sequence
  Anti-IL-6R Tocilizumab VL CDR1 variable region
RASQDISSYLN

SEQ ID NO: 129 - protein, 7 aa, Artificial Sequence
  Anti-IL-6R Tocilizumab VL CDR2 variable region
YTSRLHS

SEQ ID NO: 130 - protein, 9 aa, Artificial Sequence
  Anti-IL-6R Tocilizumab VL CDR3 variable region
QQGNTLPYT

SEQ ID NO: 131 - protein, 235 aa, Artificial Sequence
  Anti-TNF TNFR2 fusion partner
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 132 - protein, 124 aa, Artificial Sequence [sequence omitted]

SEQ ID NO: 133 - protein, 124 aa, Artificial Sequence
  Anti-B7.1/B7.2 belatacept variant CTLA-4 fusion partner
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 134 - protein, 330 aa, Artificial Sequence
  IgG1 constant heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPGK

SEQ ID NO: 135 - protein, 326 aa, Artificial Sequence
  IgG2 constant heavy chain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAQPSVFLFPPKPKDTLMISRTPEVTCVVVDVSHEDPEVQFNWYVDGXEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK